(19) United States
(12) Patent Application Publication
(10) Pub. No.: US 2009/0264388 A1
(43) Pub. Date: Oct. 22, 2009

(54) Compounds and Methods of Treating Disorders Associated With Activation of Metachromatic Cells

(75) Inventors: Karim Maghni, Laval (CA); Nadia Ouaked, Chateauguay (CA); Bertrand Lefort, Gatineau (CA); Sandra Favret, Montreal (CA)

Correspondence Address: CLARK & ELBING LLP, 101 FEDERAL STREET, BOSTON, MA, 02110, US

(73) Assignee: Valorisation Recherche HSCM, Limited Partnership, Montreal (CA)

(21) Appl. No.: 12/224,279

(22) PCT Filed: Feb. 22, 2007
(86) PCT No.: PCT/IB2007/001621

§ 371 Date: Mar. 12, 2009

Related U.S. Application Data
(60) Provisional application No. 60/775,324, filed on Feb. 22, 2006.

(51) Int. Cl. A61K 31/56

(52) U.S. Cl. 514/171

(57) ABSTRACT

The present invention relates to neurokinin-1 (NK-1) receptor antagonists in combination with an inhibitor of metachromatic cell activation, such as an anti-inflammatory agent, an immunosuppressor, or a kinase inhibitor, and use of such combinations in the treatment of disorders associated with activation of metachromatic cells. Disorders associated with the activation of metachromatic cells include allergic/non-allergic rhinitis, allergic/non-allergic asthma, allergic/non-allergic urticaria, immuno-inflammatory disorders, metachromatic cell-related autoimmune disorders, transplant rejection, and other metachromatic cell-related disorders.

### CROSS REFERENCE TO RELATED APPLICATIONS

This application claims priority from U.S. Provisional Application Ser. No. 60/775,324, filed on Feb. 22, 2006. The disclosure of which is hereby incorporated by reference.

### FIELD OF THE INVENTION

The present invention relates to neurokinin-1 (NK-1) receptor antagonists in combination with an inhibitor of metachromatic cell activation, such as an anti-inflammatory agent, an immunosuppressor, or a kinase inhibitor, or a combination thereof, and use of such combinations in the treatment of disorders associated with activation of metachromatic cells.

### BACKGROUND OF THE INVENTION

Metachromatic cells (i.e., mast cells and basophils) can be involved in antigenic and non-antigenic inflammatory responses. Allergy is one of most common manifestations of an antigenic inflammatory response (the allergen being the antigen). Diseases due to allergies provoke the infiltration of specific tissues or organs with inflammatory cells and this, together with the resulting structural changes, causes the clinical features of symptoms. Indeed, inflammation results in symptom exacerbation and is an important determinant of both current and future severity of the disease. Successful treatment of the underlying inflammatory process improves symptom profile and quality of life.

There is a need and a vast market for the development of new therapeutic strategies to block the inflammatory process and avoid the side effects of pharmacologic treatments or the risk of systemic anaphylactic reactions of allergen-specific immunotherapy. Such treatment should be safe, inexpensive, easy to administer, effective, and preferably with the capacity to interfere with the immunologic regulation of the allergic inflammation.

### SUMMARY OF THE INVENTION

The present invention features neurokinin-1 (NK-1) receptor antagonists in combination with inhibitors of metachromatic cell activation, such as an anti-inflammatory agent, an immunosuppressor, or a kinase inhibitor, and use of such combinations in the treatment of disorders associated with activation of metachromatic cells. Disorders associated with the activation of metachromatic cells include allergic/non-allergic rhinitis, allergic/non-allergic asthma, allergic/non-allergic urticaria, immuno-inflammatory disorders, metachromatic cell-related autoimmune disorders, transplant rejection, and others.

Accordingly, the first aspect of the invention features a composition containing, an NK-1 receptor inhibitor and an immunosuppressor. In desirable embodiments of the first aspect of the invention, the NK-1 receptor inhibitor is RP 67580, WIN 51078, L-733,060, L-703,606, MDL 105,212, Antagonist D, Aprepitant, R116301, CGP49823, CP-96345, CP-99994, GR-203040, MDL-103392, L-760735, SDZ-NKT-343, nolpitanitium (SR-140333), AV608, LY686017, E-6006, Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide), Vestipitant, 823296, Netupitant, H1/NK1 Dual Antagonists, MPC-4505, CP-122721, CJ-12,255, SRR240600, or TA-5538. In more desirable embodiments of the first aspect of the invention, the NK-1 receptor inhibitor is RP 67580, WIN 51078, L-733,060, L-703,606, MDL 105,212, or Aprepitant. In even more desirable embodiments of the first aspect of the invention, the NK-1 receptor inhibitor is WIN 51,708, L-703,606, L-733,060, or Aprepitant. In a yet more desirable embodiment of the first aspect of the invention, the NK-1 receptor inhibitor is Aprepitant.

In other desirable embodiments of the first aspect of the invention, the immunosuppressor inhibits immunophilin action or expression. In another desirable embodiment of the first aspect of the invention, the immunosuppressor inhibits an immunophilin-related cellular pathway. In a further desirable embodiment of the first aspect of the invention, the immunosuppressor inhibits a calcineurin. In yet another desirable embodiment of the first aspect of the invention, the immunosuppressor inhibits a calcineurin-related signaling pathway. In desirable embodiments of the first aspect of the invention, the immunosuppressor is Tacrolimus/FK506, cyclosporin A, FTY720, or rapamycin.

The second aspect of the invention features a composition containing an NK-1 receptor inhibitor and a kinase inhibitor. In desirable embodiments of the second aspect of the invention, the NK-1 receptor inhibitor is RP 67580, WIN 51078, L-733,060, L-703,606, MDL 105,212, Antagonist D, Aprepitant, R116301, CGP49823, CP-96345, CP-99994, GR-203040, MDL-103392, L-760735, SDZ-NKT-343, nolpitanitium (SR-140333), AV608, LY686017, E-6006, Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide), Vestipitant, 823296, Netupitant, H1/NK1 Dual Antagonists, MPC-4505, CP-122721, CJ-12,255, SRR240600, or TA-5538. In other desirable embodiments of the second aspect of the invention, the NK-1 receptor inhibitor is RP 67580, WIN 51078, L-733,060, L-703,606, MDL 105,212, or Aprepitant. In more desirable embodiments of the second aspect of the invention, the NK-1 receptor inhibitor is WIN 51,708, L-703,606, L-733,060, or Aprepitant. In an even more desirable embodiment of the second aspect of the invention, the NK-1 receptor inhibitor is Aprepitant.

In other desirable embodiment of the second aspect of the invention, the kinase inhibitor inhibits an FcεRI signaling pathway. In a further desirable embodiment of the second aspect of the invention, the kinase inhibitor inhibits a signaling pathway regulated by an IgE binding protein.

In an additional desirable embodiment of the second aspect of the invention, the kinase inhibitor is a syk kinase inhibitor. Desirably, the syk kinase inhibitor is BAY 61-3606. In another desirable embodiment of the second aspect of the invention, the kinase inhibitor is a Src family kinase inhibitor. Desirably, the Src family kinase inhibitor is PP1. In a further desirable embodiment of the second aspect of the invention, the kinase inhibitor is a phosphatidylinositol 3 kinase (PI3K) inhibitor. Desirably, the PI3K inhibitor is LY-294,002. In yet another desirable embodiment of the second aspect of the invention, the kinase inhibitor is a P38 mitogen-activated protein kinase (MAPK) inhibitor. Desirably, the P38 MAPK inhibitor is SB202190. In another desirable embodiment of the second aspect of the invention, the kinase inhibitor is a mitogen-activated protein kinase kinase (MAPKK) inhibitor. Desirably, the MAPKK inhibitor is PD98,059.

The third aspect of the invention features a composition containing an NK-1 receptor inhibitor and an anti-inflammatory compound, where the NK-1 receptor inhibitor is not a compound encompassed by Formula I (as set forth herein), Aprepitant, or Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide).

In a desirable embodiment of the third aspect of the invention, the NK-1 receptor inhibitor is RP 67580, WIN 51078, L-733,060, L-703,606, MDL 105,212, Antagonist D, R116301, CGP49823, CP-96345, CP-99994, GR-203040, MDL-103392, L-760735, SDZ-NKT-343, nolpitanitium (SR-140333), AV608, LY686017, E-6006, Vestipitant, 823296, Netupitant, H1/NK1 Dual Antagonists, MPC-4505, CP-122721, CJ-12,255, SRR240600, or TA-5538. In more desirable embodiments of the third aspect of the invention, the NK-1 receptor inhibitor is RP 67580, WIN 51078, L-733,060, L-703,606, or MDL 105,212. In yet more desirable embodiments of the third aspect of the invention, the NK-1 receptor inhibitor is WIN 51,708, L-703,606, or L-733,060.

In other desirable embodiments of the third aspect of the invention, the anti-inflammatory compound is a steroid. Desirably, the steroid is dexamethasone, fluticasone, flunisolide, budesonide, or mometasone.

The fourth aspect of the invention features a composition containing (i) an NK-1 receptor inhibitor, (ii) an inhibitor of metachromatic cell activation, and (iii) a beta-2 adrenergic receptor agonist. In a desirable embodiment of the fourth aspect of the invention, the inhibitor of metachromatic cell activation is fluticasone and the beta-2 adrenergic receptor agonist is salmeterol. In another desirable embodiment of the fourth aspect of the invention, the inhibitor of metachromatic cell activation is budesonide and the beta-2 adrenergic receptor agonist is formoterol. In a further desirable embodiment of the fourth aspect of the invention, the inhibitor of metachromatic cell activation is mometasone and the beta-2 adrenergic receptor agonist is indacaterol.

The fifth aspect of the invention features a composition containing (i) an NK-1 receptor inhibitor, (ii) an anti-inflammatory compound, and (iii) a kinase inhibitor. The sixth aspect of the invention features a composition containing (i) an NK-1 receptor inhibitor, (ii) an anti-inflammatory compound, and (iii) an immunosuppressor. In a desirable embodiment of the sixth aspect of the invention, the anti-inflammatory compound is fluticasone and the immunosuppressor is Tacrolimus/FK506. In another desirable embodiment of the sixth aspect of the invention, the anti-inflammatory compound is fluticasone and the immunosuppressor is cyclosporin A. In a further desirable embodiment of the sixth aspect of the invention, the anti-inflammatory compound is budesonide and the immunosuppressor is Tacrolimus/FK506. In yet another desirable embodiment of the sixth aspect of the invention, the anti-inflammatory compound is budesonide and the immunosuppressor is cyclosporin A.

The seventh aspect of the invention features a composition containing (i) an NK-1 receptor inhibitor, (ii) an immunosuppressor, and (iii) a kinase inhibitor. In desirable embodiments of the fifth or the seventh aspect of the invention the kinase inhibitor is BAY61-3606, PP1, LY-294,002, SB202190, or PD98,059. In desirable embodiments of the fourth, fifth, sixth, or seventh aspect of the invention, the NK-1 receptor inhibitor is Aprepitant.

In desirable embodiments of any one of the first seven aspects of the invention, the composition is in a pharmaceutically acceptable carrier. In another aspect, the invention features a pharmaceutically acceptable composition containing the composition of any one of the first seven aspects of the invention. In a further aspect, the invention features a kit containing the composition of any one of the first seven aspects of the invention and instructions for administration of the composition to a subject.

In the eighth aspect, the invention features a method of treating a disease, disorder, or condition associated with metachromatic cell activation in a subject. This method involves administering to a subject in need thereof a therapeutically effective amount of a composition containing an NK-1 receptor inhibitor and an immunosuppressor. In a desirable embodiment of the eighth aspect of the invention, the disease, disorder, or condition associated with metachromatic cell activation is transplant rejection.

In other desirable embodiments of the eighth aspect of the invention, the NK-1 receptor inhibitor is RP 67580, WIN 51078, L-733,060, L-703,606, MDL 105,212, Antagonist D, Aprepitant, R116301, CGP49823, CP-96345, CP-99994, GR-203040, MDL-103392, L-760735, SDZ-NKT-343, nolpitanitium (SR-140333), AV608, LY686017, E-6006, Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide), Vestipitant, 823296, Netupitant, H1/NK1 Dual Antagonists, MPC-4505, CP-122721, CJ-12,255, SRR240600, or TA-5538. In more desirable embodiments of the eighth aspect of the invention, the NK-1 receptor inhibitor is RP 67580, WIN 51078, L-733,060, L-703,606, MDL 105,212, or Aprepitant. In yet more desirable embodiments of the eighth aspect of the invention, the NK-1 receptor inhibitor is WIN 51,708, L-703,606, L-733,060, or Aprepitant. In even more desirable embodiments of the eighth aspect of the invention, the NK-1 receptor inhibitor is Aprepitant.

In other desirable embodiments of the eighth aspect of the invention, the immunosuppressor inhibits immunophilin action or expression. In another desirable embodiment of the eighth aspect of the invention, the immunosuppressor inhibits an immunophilin-related cellular pathway. In an additional desirable embodiment of the eighth aspect of the invention, the immunosuppressor inhibits a calcineurin. In yet another desirable embodiment of the eighth aspect of the invention, the immunosuppressor inhibits a calcineurin-related signaling pathway. In further desirable embodiments of the eighth aspect of the invention, the immunosuppressor is Tacrolimus/FK506, cyclosporin A, FTY720, or rapamycin.

In another desirable embodiment of the eighth aspect of the invention, the composition further contains an anti-inflammatory compound. Desirably, the anti-inflammatory compound is dexamethasone, fluticasone, flunisolide, budesonide, or mometasone. In yet another desirable embodiment of the eighth aspect of the invention, the composition further contains a kinase inhibitor. Desirably, the kinase inhibitor is BAY61-3606, PP1, LY-294,002, SB202190, or PD98,059.

In the ninth aspect, the invention features a method of treating a disease, disorder, or condition associated with metachromatic cell activation in a subject. This method involves administering to a subject in need thereof a therapeutically effective amount of a composition containing an NK-1 receptor inhibitor and a kinase inhibitor.

In desirable embodiments of the ninth aspect of the invention, the NK-1 receptor inhibitor is RP 67580, WIN 51078, L-733,060, L-703,606, MDL 105,212, Antagonist D, Aprepitant, R116301, CGP49823, CP-96345, CP-99994, GR-203040, MDL-103392, L-760735, SDZ-NKT-343, nolpitanitium (SR-140333), AV608, LY686017, E-6006, Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide), Vestipitant, 823296, Netupitant, H1/NK1 Dual Antagonists, MPC-4505, CP-122721, CJ-12,255, SRR240600, or TA-5538. In more desirable embodiments of the ninth aspect of the invention, the NK-1 receptor inhibitor is RP 67580, WIN 51078, L-733,060, L-703,606, MDL 105,212, or Aprepitant. In even more desirable embodiments of the ninth aspect of the invention, the NK-1 receptor inhibitor is WIN 51,708, L-703,606, L-733,060, or Aprepitant. In an even more desirable embodiment of the ninth aspect of the invention, the NK-1 receptor inhibitor is Aprepitant.

In another desirable embodiment of the ninth aspect of the invention, the kinase inhibitor inhibits an FcεRI signaling pathway. In a further desirable embodiment of the ninth aspect of the invention, the kinase inhibitor inhibits a signaling pathway regulated by an IgE binding protein. In an additional desirable embodiment of the ninth aspect of the invention, the kinase inhibitor is a syk kinase inhibitor. Desirably, the syk kinase inhibitor is BAY 61-3606. In a further desirable embodiment of the ninth aspect of the invention, the kinase inhibitor is a Src family kinase inhibitor. Desirably, the Src family kinase inhibitor is PP1. In yet another desirable embodiment of the ninth aspect of the invention, the kinase inhibitor is a phosphatidylinositol 3 kinase (PI3K) inhibitor. Desirably, the PI3K inhibitor is LY-294,002. In a further desirable embodiment of the ninth aspect of the invention, the kinase inhibitor is a P38 mitogen-activated protein kinase (MAPK) inhibitor. Desirably, the P38 MAPK inhibitor is SB202190. In yet a further desirable embodiment of the ninth aspect of the invention, the kinase inhibitor is a mitogen-activated protein kinase kinase (MAPKK) inhibitor. Desirably, the MAPKK inhibitor is PD98,059.

In another desirable embodiment of the ninth aspect of the invention, the composition further contains an anti-inflammatory compound. Desirably, the anti-inflammatory compound is dexamethasone, fluticasone, flunisolide, budesonide, or mometasone. In other desirable embodiments of the ninth aspect of the invention, the composition further contains an immunosuppressor. Desirably, the immunosuppressor is Tacrolimus/FK506, cyclosporin A, FTY720, or rapamycin.

The tenth aspect of the invention features a method of treating a disease, disorder, or condition associated with metachromatic cell activation in a subject. This method involves administering to a subject in need thereof a therapeutically effective amount of a composition containing an NK-1 receptor inhibitor and an inhibitor of metachromatic cell activation, where the NK-1 receptor inhibitor is not a compound encompassed by Formula I (as described herein), or Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide).

In a desirable embodiment of the tenth aspect of the invention, the NK-1 receptor inhibitor is RP 67580, WIN 51078, L-733,060, L-703,606, MDL 105,212, Antagonist D, R116301, CGP49823, CP-96345, CP-99994, GR-203040, MDL-103392, L-760735, SDZ-NKT-343, nolpitanitium (SR-140333), AV608, LY686017, E-6006, Vestipitant, 823296, Netupitant, H1/NK1 Dual Antagonists, MPC-4505, CP-122721, CJ-12,255, SRR240600, or TA-5538. In more desirable embodiments of the tenth aspect of the invention, the NK-1 receptor inhibitor is RP 67580, WIN 51078, L-733,060, L-703,606, or MDL 105,212. In yet more desirable embodiments of the tenth aspect of the invention, the NK-1 receptor inhibitor is WIN 51,708, L-703,606, or L-733,060.

In other desirable embodiments of the tenth aspect of the invention, the inhibitor of metachromatic cell activation is an anti-inflammatory compound. Desirably, the anti-inflammatory compound is a steroid, and, in desirable embodiments, is dexamethasone, fluticasone, flunisolide, budesonide, or mometasone.

In the eleventh aspect, the invention features a method of treating a disorder selected from allergic or non-allergic rhinitis, allergic or non-allergic asthma, allergic or non-allergic urticaria, an immuno-inflammatory disorder, an autoimmune disorder, and transplant rejection in a subject. This method involves administering to a subject in need thereof a therapeutically effective amount of a composition containing an NK-1 receptor inhibitor and an inhibitor of metachromatic cell activation, where the NK-1 receptor inhibitor is not a compound encompassed by Formula I (as described herein).

In desirable embodiments of the eleventh aspect the disorder is allergic or non-allergic rhinitis. In other desirable embodiments of the eleventh aspect of the invention, the disorder is allergic or non-allergic asthma. In additional desirable embodiments of the eleventh aspect of the invention, the disorder is allergic or non-allergic urticaria. In further desirable embodiments of the eleventh aspect of the invention, the disorder is an autoimmune disorder. In yet another desirable embodiment of the eleventh aspect of the invention, the disorder is transplant rejection. In a further desirable embodiment of the eleventh aspect of the invention, the disorder is an immuno-inflammatory disorder.

In additional desirable embodiments of the eleventh aspect of the invention, the NK-1 receptor inhibitor is RP 67580, WIN 51078, L-733,060, L-703,606, MDL 105,212, Antagonist D, Aprepitant, R116301, CGP49823, CP-96345, CP-99994, GR-203040, MDL-103392, L-760735, SDZ-NKT-343, nolpitanitium (SR-140333) AV608, LY686017, E-6006, Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide), Vestipitant, 823296, Netupitant, H1/NK1 Dual Antagonists, MPC-4505, CP-122721, CJ-12,255, SRR240600, or TA-5538. In more desirable embodiments of the eleventh aspect of the invention, the NK-1 receptor inhibitor is RP 67580, WIN 51078, L-733,060, L-703,606, MDL 105,212, or Aprepitant. In even more desirable embodiments of the eleventh aspect of the invention, the NK-1 receptor inhibitor is WIN 51,708, L-703,606, L-733,060, or Aprepitant. In yet more desirable embodiments of the eleventh aspect of the invention, the NK-1 receptor inhibitor is Aprepitant.

In further desirable embodiments of the eleventh aspect of the invention, the inhibitor of metachromatic cell activation is an anti-inflammatory compound. Desirably, the anti-inflammatory compound is dexamethasone, fluticasone, flunisolide, budesonide, or mometasone. In other desirable embodiments of the eleventh aspect of the invention, the inhibitor of metachromatic cell activation is an immunosuppressor or a kinase inhibitor.

In desirable embodiments of the eighth, ninth, tenth, or eleventh aspect of the invention, the subject is a mammal. Desirably, the mammal is a human. In other desirable embodiments of the eighth, ninth, tenth, or eleventh aspect of the invention the composition is in a pharmaceutically acceptable carrier.

In addition, the compositions described herein (including the compositions of the first seven aspects of the invention) may be used in the manufacture of a medicament for the treatment of the diseases, disorders, or conditions associated with metachromatic cell activation described herein.

### DEFINITIONS

“NK-1 receptor” as used herein refers to a receptor that binds Substance P (an 11 amino acid polypeptide with the sequence: Arg Pro Lys Pro Gln Gln Phe Phe Gly Leu Met (SEQ ID NO: 1)). Desirably, an NK-1 receptor is a human neurokinin-1 receptor. Other desirable NK-1 receptors include spliced isoforms (i.e., short and long isoforms) and other isoforms of the neurokinin-1 (NK-1) receptor, including the reported single nucleotide polymorphism (SNPs) isoforms of the NK-1 receptor (e.g., GenBank Accession number BD223571. Human NK-1 receptor sequences are deposited under GenBank accession numbers NM_{—}001058 (long isoform) and NM_{—}015727 (short isoform).

An “inhibitor of metachromatic cell activation” as used herein refers to a compound that decreases a biological activity of a metachromatic cell indicative of its activation. Activation of metachromatic cells involves degranulation which can be assayed using the methods described herein. For example, the activation of metachromatic cells may be determined by quantifying the release of β-hexosaminidase as a marker of cell activation and degranulation. In addition, degranulation itself is a marker of metachromatic cell activation. Degranulation involves, for example, the release of IL-4 secretory granules or basic protein secretory granules and can be assayed using standard methods in the art. Degranulation may be induced by IgE-dependent and IgE-independent stimuli. IgE-independent stimuli desirably include the activation of a calcium ionophore. Desirably, an inhibitor decreases activation of a metachromatic cell by 10%, 20%, 30%, 40%, 50%, 70%, 80%, 90%, or even 100% relative to a control. Exemplary inhibitors of metachromatic cell activation include anti-inflammatory compounds, immunosuppressors, and kinase inhibitors as defined herein. In desirable embodiments the inhibitor of metachromatic cell activation is a glucocorticoid, 5-[2-(5,6-Diethyl-2,3-dihydro-1H-inden-2-ylamino)-1(R)-hydroxyethyl]-8-hydroxychinolin-2(1H)-on), cromoglycate, a methylxanthin, an anti-histaminic, a beta-2 adrenergic receptor agonist, a leucotriene antagonist, or a combination of such compounds.

By a “beta-2 adrenergic receptor agonist” as used herein is a compound that results in beta-2 adrenergic receptor activation. Desirably, a beta-2 adrenergic receptor agonist causes muscle relaxation and/or vasodilation. In desirable embodiments, the beta2 adrenergic receptor agonist is QAB-149 (indacaterol; 5-[2-(5,6-Diethyl-2,3-dihydro-1H-inden-2-ylamino)-1(R)-hydroxyethyl]-8-hydroxychinolin-2(1H)-on). Other desirable beta-2 adrenergic receptor agonists include salbutamol (albuterol), levalbuterol, terbutaline, pirbuterol, procaterol, metaproterenol, fenoterol, bitolerol mesylate, salmeterol, formoterol, and bambuteral.

A “disease, disorder, or condition associated with metachromatic cell activation” as used herein refers to any disease, disorder, or condition in which metachromatic cells are abnormally activated, or an injury which results in metachromatic cell activation. Desirably, the disease, disorder, or condition is a disease of the upper and lower respiratory tract, for example, bronchial asthma, allergic asthma, non-allergic asthma, lymphomatous tracheobronchitis, allergic hypersensitivity or a hypersecretion condition, such as chronic bronchitis and cystic fibrosis; pulmonary fibrosis of various aetiologies (e.g., idiopathic pulmonary fibrosis), chronic obstructive pulmonary disease (COPD), sarcoidosis, allergic and non-allergic rhinitis; allergic or non-allergic urticaria; a skin-related diseases characterized by deregulated inflammation, tissue remodeling, angiogenesis, and neoplasm, a disease of the gastrointestinal tract, such as Crohn's disease, Hirschsprung's disease, diarrhea, malabsorption conditions, and inflammatory conditions; a disorder of the central and peripheral nervous system, such as depression, anxiety states, Parkinson's disease, migraine and other forms of cranial pain, strokes, emesis; a disease of the immune system, such as in the splenic and lymphatic tissues, an autoimmune disease or other immune-related diseases; a disease of the cardiovascular system, such as pulmonary edema, hypertension, atherosclerosis, pre-eclampsia, complex regional pain syndrome type 2, stroke and chronic inflammatory diseases such as arthritis, a bone-related diseases such as rheumatoid arthritis, as well as pain, chronic pain such as fibromyalgia, and other disorders in which the action of neurokinins, tachykinins or other related substances (e.g., hemokinins) are involved in the pathogenesis, pathology, and aetiology.

Additional examples of disorders associated with metachromatic cell activation include acne vulgaris; acute respiratory distress syndrome; Addison's disease; allergic intraocular inflammatory diseases, ANCA-associated small-vessel vasculitis; ankylosing spondylitis; atopic dermatitis; autoimmune hemolytic anemia; autoimmune hepatitis; Behcet's disease; Bell's palsy; bullous pemphigoid; cerebral ischaemia; cirrhosis; Cogan's syndrome; contact dermatitis; Cushing's syndrome; dermatomyositis; diabetes mellitus; discoid lupus erythematosus; lupus nephritis; eosinophilic fasciitis; erythema nodosum; exfoliative dermatitis; focal glomerulosclerosis; focal segmental glomerulosclerosis; segmental glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic thrombocytopenic purpura; immune thrombocytopenic purpura inflammatory bowel or gastrointestinal disorders, inflammatory dermatoses; lichen planus; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; septic shock syndrome; shoulder tendinitis or bursitis; Sjogren's syndrome; Still's disease, Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis.

“Metachromatic cells” as used herein are a basophils or mast cells.

“NK-1 receptor inhibitor” or “NK-1 receptor antagonist” as used herein refers to a compound that directly or indirectly decreases the biological activity of an NK-1 receptor. An NK-1 inhibitor desirably decreases the biological activity of an NK-1 receptor by inhibiting a signaling pathway regulated by a protein bound by Substance P or by decreasing the expression or activity of a protein that binds Substance P. Desirably, an NK-1 receptor biological activity is reduced by 10%, 20%, 30%, 40%, 50%, 70%, 80%, 90%, or even 100% relative to a control. The activation of NK-1 receptor may be assayed through the measurement of intracellular signaling pathway such as changes in intracellular calcium level, production of IP3 (inositol tripbosphate), activation of transcription factor (e.g., NF-kappaB), foot tapping in the gerbil (Kramer et al., Science 281:1642-1645 (1998); Duffy et al., JPET 301:536-542, 2002), etc., using standard methods in the art. Exemplary desirable NK-1 inhibitors include RP 67580, WIN 51078, L-733,060, L-703,606, MDL 105,212, Antagonist D, Aprepitant, R116301, CGP49823, CP-96345, CP-99994, GR-203040, MDL-103392 (racemate of the active enantiomer MDL-105212), L-760735, SDZ-NKT-343, nolpitanitium (SR-140333), the 1-aryl-2-acylamino-ethane compounds described in U.S. Pat. No. 5,929,067, E-6006 (5-(alpha-[2-(dimethylamino)ethoxy]-2-thienylmethyl)-1-methyl-1H-pyrazole), Vestipitant (2S)-N-{(1R)-1-[3,5-bis(trifluoromethyl)phenyl]ethyl}-2-(4-fluoro-2-methylphenyl)-N-methylpiperazine-1-carboxamide), Netupitant (2-(3,5-Bis.trifluoromethyl-phenyl)-N-methyl-N-[6-(4-methyl-piperazin-1-yl)-4-0-tolyl-pyridin-3-yl]-isobutyramide; C₃₀H₃₂F₆N₄O), and Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide) (as described herein, for example, in Table 1). In desirable embodiments, the NK-1 receptor inhibitor is WIN 51,708, L-703,606 oxalate salt, L-733,060 hydrochloride, or Aprepitant.

In some embodiments of the invention, compounds of Formula I as provided below (and as disclosed in U.S. Patent Application Publication No. US 2003/0158173) are specifically excluded from the definition of an NK-1 receptor inhibitor or antagonist.

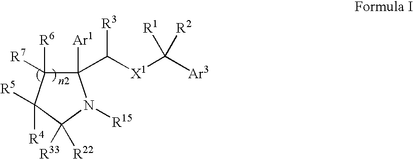

Ar¹ and Ar² are each independently selected from the group consisting of R¹⁷-heteroaryl and

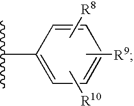

X¹ is —O—, —S—, —SO—, —SO₂—, —NR³⁴—, —N(COR¹²)— or —N(SO₂R¹⁵); when X¹ is —SO—, —SO₂—, —N(COR¹²)— or —N(SO₂R¹⁵)—, then: R¹ and R² are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a chemically feasible C₃ to C₆ alkylene ring; or when X¹ is —O—, —S— or —NR³⁴—, then: R¹ and R²— are each independently selected from the group consisting of H, C₁-C₆ alkyl, hydroxy(C₁-C₃alkyl), C₃-C₈ cycloalkyl, —CH₂F, —CHF₂ and —CF₃; or R¹ and R², together with the carbon atom to which they are both attached, form a chemically feasible C₃ to C₆ alkylene ring; or R¹ and R², together with one another and the carbon atom to which they are both attached, form a C═O group; R³ is selected from the group consisting of H, C₁-C₆ alkyl, hydroxy(C₁-C₃ alkyl), C₃-C₈ cycloalkyl, —CH₂F, —CHF₂ and —CF₃; each R⁶ is independently selected from the group consisting of H, C₁-C₆ alkyl and —OH; each R⁷ is independently selected from the group consisting of H and C₁-C₆ alkyl; n₂ is 1 to 4; R⁴ and R⁵ are each independently selected from the group consisting of —(CR²⁸R²⁹)ₙ₁-G, where, n₁ is 0 to 5; and G is H, —CF₃, —CHF₂, —CH₂F, —OH, —O—(C₁-C₆ alkyl), —OCH₂F, —OCHF₂, —OCF₃, —OCH₂CF₃, —O—(C₃-C₉ cycloalkyl), —O—(C₁-C₆)alkyl(C₃-C₈ cycloalkyl), —NR¹³R¹⁴, —SO₂NR¹³R¹⁴, —NR¹²SO₂R¹³, —NR¹²C(O)R¹⁴, —NR¹²C(O)OR¹³, —NR¹²(C(O)NR¹³R¹⁴), —C(O)NR¹³R¹⁴, —C(O)OR¹³, —C₃-C₈ cycloalkyl, (R¹⁹)ᵣ-aryl, (R¹⁹)ᵣ-heteroaryl, —OC(O)R¹⁴, —OC(O)N¹³R¹⁴, —C(═NOR¹⁴)(R¹³), —C(O)R¹³, —C(OR¹²)(R¹³)(R¹⁴), heterocycloalkenyl optionally substituted by 1 to 4 substituents independently selected from the group consisting of R³⁰ and R³¹,

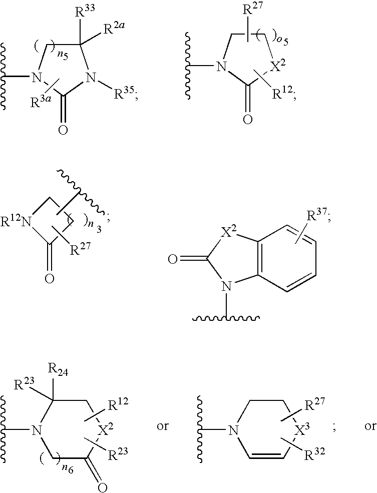

R⁴ and R⁵ together are ═O, ═NOR¹²; or R⁴ and R⁵, together with the carbon atom to which they are both attached, form a chemically feasible 4- to 8-membered heterocycloalkyl or heterocycloalkenyl ring containing 1 to 3 groups independently selected from X², provided that at least one X² is —NR³⁵—, —O—, —S—, —S(O)— or —SO₂—, the chemically feasible ring being optionally substituted with from 1 to 6 substituents independently selected from the group consisting of R³⁰ and R³¹; provided that R⁴ and R⁵ are not both selected from the group consisting of H, alkyl, and cycloalkyl; further provided that, when one of R⁴ and R⁵ is —OH, then the other one of R⁴ and R⁵ is not alkyl or (R¹⁹)ᵣ-aryl; R⁸, R⁹, and R¹⁰ are each independently selected from the group consisting of H, C₁-C₆ alkyl, C₃-C₈ cycloalkyl, —OR¹², halogen, —CN, —NO₂, —CF₃, —CHF₂, —CH₂F, —CH₂CF₃, —OCF₃, —OCHF₂, —OCH₂F, —OCH₂CF₃, —COOR¹², —CONR²¹R²², —OC(O)NR²¹R²², —OC(O)R¹², —NR²¹COR¹², —NR²¹CO₂R¹⁵, —NR²¹CONR²¹R²², —NR²¹SO₂R¹⁵, —NR²¹R²², —SO₂NR²¹R²², —S(O)ₙ₆R¹⁵, (R¹⁹)ᵣ-aryl and (R¹⁹)ᵣ-heteroaryl; R¹² is H, C₁-C₆ alkyl or C₃-C₈ cycloalkyl; R¹³ and R¹⁴ are each independently selected from the group consisting of H, C₁-C₆ alkyl, C₃-C₈ cycloalkyl, (C₃-C₈)cycloalkyl(C₁-C₆)alkyl, —CH₂CF₃, aryl and heteroaryl; or R¹³ and R¹⁴, together with the nitrogen atom to which they are both attached, form a chemically feasible 4- to 7-membered saturated or unsaturated ring that is optionally substituted with —OR¹², where one of the carbon atoms in the ring is optionally replaced by a heteroatom selected from the group consisting of —O—, —S— and —NR³⁴—; n₆ is 0, 1 or 2; R¹⁵ is C₁-C₆ alkyl, C₃-C₈ cycloalkyl, —CF₃ or —CH₂CF₃; R¹⁸ is H, C₁-C₆ alkyl, C₃-C₈ cycloalkyl, (C₃-C₈)cycloalkyl(C₁-C₆)alkyl, hydroxy(C₂-C₆)alkyl or —P(O)(OH)₂; each R¹⁹ is a substituent on the aryl or heteroaryl ring to which it is attached, and is independently selected from the group consisting of H, C₁-C₆ alkyl, C₃-C₈ cycloalkyl, C₁-C₆ alkoxy, —OH, halogen, —CN, —NO₂, —CF₃, —CHF₂, —CH₂F, —OCF₃, —OCHF₂, —OCH₂F, —O—(C₁-C₆ alkyl), —O—(C₃-C₈ cycloalkyl), —COOR¹², —CONR²¹R²², —OC(O)NR²¹R²², —OC(O)R¹², —NR²¹R²², —NR²¹COR¹², NR²¹CO₂R¹², —NR²¹CONR²¹R²², NR²¹SO₂R¹⁵ and —S(O)ₙ₆R¹⁵; R²¹ and R²² are each independently selected from the group consisting of H, C₁-C₆ alkyl, C₃-C₈ cycloalkyl, and benzyl; or R²¹ and R²², together with the nitrogen atom to which they are both attached, form a chemically feasible 4- to 7-membered saturated or unsaturated ring, where one of the carbon atoms in the ring is optionally replaced by a heteroatom selected from the group consisting of —O—, —S—, and —NR³⁴—; R²³ and R²⁴ are each independently selected from the group consisting of H and C₁-C₆ alkyl; or R²³ and R²⁴, together with the carbon atom to which they are both attached, form a C═O or cyclopropyl group; R²⁷ is H, —OH or C₁-C₆ alkyl; R²⁸ and R²⁹ are each independently selected from the group consisting of H and C₁-C₂ alkyl; R³⁰ and R³¹ are each independently selected from the group consisting of H, —OH, C₁-C₆ alkyl C₃-C₈ cycloalkyl, (C₃-C₈)cycloalkyl(C₁-C₆)alkyl and —C(O)NR¹³R¹⁴; or R³⁰ and R³¹, together with the carbon atom to which they are both attached, form ═O, ═S, a cyclopropyl ring or ═NR³⁶; R³² and R³³ are each independently selected from the group consisting of H and C₁-C₆ alkyl; R³⁴ is H, C₁-C₆ alkyl, C₃-C₈ cycloalkyl, (C₃-C₈)cycloalkyl(C₁-C₆)alkyl or hydroxy(C₂-C₆)alkyl; R³⁵ is H, C₁-C₆ alkyl, C₃-C₈ cycloalkyl, (C₃-C₈)cycloalkyl(C₁-C₆)alkyl, —P(O)(OH)₂, allyl, hydroxy(C₂-C₆)alkyl, (C₁-C₆)alkoxy(C₁-C₆)alkyl, —SO₂R¹⁵, or —(CH₂)₂—N(R¹²)—SO₂—R¹⁵; R³⁶ is H, C₁-C₆ alkyl, C₃-C₈ cycloalkyl, (C₃-C₈)cycloalkyl(C₁-C₆)alkyl, —NO₂, —CN or OR¹²; R³⁷ is 1 to 3 substituents independently selected from the group consisting of H, C₁-C₆ alkyl, —OH, C₁-C₆ alkoxy and halogen; r is 1 to 3; X² is —NR³⁵—, —O—, —S—, —S(O)—, —SO₂—, —CH₂—, —CF₂— or —CR¹²F—; X³ is —NR³⁴, —N(CONR¹³R¹⁴)—, —N(CO₂R¹³)—, —N(SO₂R¹⁵)—, —N(COR¹²)—, —N(SO₂NHR¹³)—, —O—, —S—, —S(O)—, —SO₂—, —CH₂—, —CF₂— or —CR¹²F—; n₃ is 1 to 5; and n₅ is 1 to 3.

In other embodiments Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide) is specifically excluded from the definition of an NK-1 receptor inhibitor or antagonist.

In yet other embodiments, Aprepitant (structure provided herein) is specifically excluded from the definition of an NK-1 receptor inhibitor or antagonist.

Indirect inhibition of an NK-1 receptor includes, for example, capturing the receptor agonists (tachykinins such as Substance P, and related molecules such as hemokinins and endokinins) by using ligands like monoclonal antibodies or any molecules having an affinity for the agonists and hindering the natural association between an agonist and the NK-1 receptor. Inhibition also includes methods aimed at inducing the degradation of Substance P and related molecules such as hemokinins and endokinins into biological inactive substances, which would reduce the amount of active agonist. Further, indirect inhibition may involve inhibition of the activity of a component in an NK-1 receptor signaling pathway. Direct inhibition of an NK-1 receptor includes, for example, ligands that bind directly to the receptor or Substance P binding-protein and reduce or inhibit its activity. Monoclonal antibodies, and competitive and non-competitive pharmaceutical antagonists are examples of a direct inhibition.

An “anti-inflammatory” compound as used herein refers to a compound that reduces inflammation in a subject. Desirably, an anti-inflammatory compound decreases metachromatic cell activation. In desirable embodiments an anti-inflammatory compound is a steroid, such as a glucocorticoid. Desirably, the glucocorticoid is 11-alpha, 17-alpha,21-trihydroxypregn-4-ene-3,20-dione; 11-beta, 16-alpha, 17,21-tetrahydroxypregn-4 Åe-3,20-dione; 11-beta,16-alpha,17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclometasone; aldosterone; algestone; alphaderm; amadinone; amcinonide; anagestone; androstenedione; anecortave acetate; beclomethasone; betamethasone; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasone; clobetasol propionate; clocortolone; clocortolone pivalate; clogestone; cloprednol; corticosterone; Cortisol; Cortisol acetate; Cortisol butyrate; Cortisol cypionate; Cortisol octanoate; Cortisol sodium phosphate; Cortisol sodium succinate; Cortisol valerate; 21-deoxycortisol; cortisone; cortisone acetate; cortivazol; cortodoxone; daturaolone; deflazacort; dehydroepiandrosterone; delmadinone; deoxycorticosterone; deprodone; descinolone; desonide; desoximetasone; dexafen; dexamethasone; dexamethasone 21-acetate; dexamethasone sodium phosphate; dichlorisone; diflorasone; diflorasone diacetate; diflucortolone; dihydroelatericin a; domoprednate; doxibetasol; ecdysone; ecdysterone; endrysone; enoxolone; flucinolone; fludrocortisone; fludrocortisone acetate; flugestone; flumethasone; flumethasone pivalate; flumoxonide; flunisolide; fluocinolone acetonide; fluocinolone; 9-fluorocortisone; fluocinonide; fluocortolone; fluorohydroxyandrostenedione; fluorometholone; fluorometholone acetate; fluoxymesterone; fluprednidene; fluprednisolone; flurandrenolide; flurandrenolone; fluticasone; fluticasone propionate; formebolone; formestane; formocortal; gestonorone; glyderinine; halcinonide; halometasone; halopredone; haloprogesterone; hydrocortisone cypionate; hydrocortisone 21-butyrate; hydrocortisone aceponate; hydrocortisone acetate; hydrocortisone buteprate; hydrocortisone butyrate; hydrocortisone; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; hyrcanoside; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone triamcinolone acetonide; triamcinolone acetonide 21-palmitate; triamcinolone diacetate; triamcinolone hexacetonide; trimegestone; turkesterone; or wortmannin.

More desirably, the glucocorticoid is dexamethasone, fluticasone, flunisolide, budesonide, or a combination of two or more glucocorticoids. Other desirable anti-inflammatory compounds are agents that inhibit the action or expression of endogenous inhibitors of glucocorticoid action or expression (e.g., by inhibiting expression of the beta-isoform of the glucocorticoid receptor using RNAi). Desirably, inflammation is reduced by 10%, 20%, 30%, 40%, 50%, 70%, 80%, 90%, or even 100% relative to a control as measured, for example, by metachromatic cell activation.

“Immunosuppressor” or “Immunosuppressive agent” as used herein is a compound that decreases an immune response in a subject. Desirably, an immunosuppressor or immunosuppressive agent decreases metachromatic cell activation. Desirably, the immunosuppressor is a compound acting on immunophilins. Examples of compounds acting on immunophilins include Tacrolimus, Rapamycin, Cyclosporin A, cytostatics such as alkylating agents (e.g., cyclophosphamide, nitrosoureas, platinium compounds, etc.), antimetabolites (e.g., methotrexate, azathioprine, mercaptopurine, etc.), and cytotoxic antibiotics (dactinomycin, anthracyclin, bleomycin, mithramycin, etc.), polyclonal antibodies (Atgam®, etc.), monoclonal antibody (OKT3 (Ortho Biotech), etc.), and other drugs such as interferons, TNF binding proteins, mycophenolate, and small biological agents (e.g., FTY720 (fingolimod), etc.). In other desirable embodiments, an immunosuppressor inhibits immunophilin action or expression or inhibits an immunophilin-related cellular pathway (e.g., by inhibiting the Nuclear Factor of Activated T cells (NFAT)-calcineurin pathway). Desirably, the immune response is reduced by 10%, 20%, 30%, 40%, 50%, 70%, 80%, 90%, or even 100% relative to a control as measured, for example, by metachromatic cell activation.

A “kinase inhibitor” as used herein is a compound that decreases the activity of a membrane bound or cytoplasmic protein tyrosine or serine/threonine kinase. Desirably, the kinase is Syk, ZAP-70, a member of the Src family (e.g., Lyn, Fyn, etc.), phosphatidylinositol 3-kinase (PI3K), p38 MAP kinase, or mitogen-activated protein kinase kinase (MAPKK). Desirably, a kinase inhibitor decreases the activity of a kinase by 10%, 20%, 30%, 40%, 50%, 70%, 80%, 90%, or even 100% relative to a control as measured, for example; by metachromatic cell activation. Desirable kinase inhibitors include the Syk kinase inhibitor BAY61-3606, the Src family kinase inhibitor PP1, the PI3K inhibitor LY-294,002, the p38 MAP kinase inhibitor SB202190, and the MAPKK inhibitor PD98,059. Kinase inhibition may be determined by measuring inhibition of an FcεRI receptor signaling pathway or a signaling pathway regulated by a receptor that bings IgE.

An “immuno-inflammatory disorder” as used herein is a disease, disorder, or condition associated with antigenic and/or non-antigenic activation of cells expressing a protein that binds IgE, such as metachromatic cells. The local immuno-inflammatory allergic disorder desirably is allergy, asthma, rhinitis, eczema, urticaria, contact dermatitis, otitis media, conjunctiva or headaches. The local immuno-inflammatory allergic disorder desirably is anaphylaxis.

An “autoimmune disorder” as used herein refers to a disorder resulting from attack of a body's own tissue by its immune system. Desirably, the autoimmune disease is diabetes melitus, multiple sclerosis, premature ovarian failure, scleroderma, Sjogren's disease, lupus, alopecia (baldness), polyglandular failure, Grave's disease, hypothyroidism, polymyosititis, Celiac disease, Crohn's disease, inflammatory bowel disease, ulcerative colitis, autoimmune hepatitis, hypopituitarism, Guillain-Barre syndrome, myocardititis, Addison's disease, autoimmune skin diseases, uveititis, pernicious anemia, polymyalgia rheumatica, Goodpasture's syndrome, hypoparathyroidism, Hashimoto's thyoriditis, Raynaud's phenomenon, polymyaglia rheumatica, and rheumatoid arthritis.

### ADVANTAGES

One limit of current asthma therapy are the side-effects related to the use of glucocorticoids, and the risk of enhanced side-effects is related to the requirement of increasing glucocorticoid doses to reduce or control asthma symptoms.

For instance, subcutaneous immunotherapy represents the standard immunotherapy care of allergic patients. Some clinical studies have documented the efficacy of such treatment, and others have reported that the magnitude of efficacy is equivalent to pharmacologic treatment. The limits of subcutaneous immunotherapy include (i) the risk of inducing systemic anaphylactic reactions and (ii) the incomplete level of evidence for the long-term efficacy and the preventive capacity of subcutaneous immunotherapy.

Local immunotherapy (sublingual immunotherapy) as opposed systemic immunotherapy may reduce the risk of inducing anaphylactic reactions. However, the long-term efficacy (clinical effect persistence after terminating treatment), and the preventive capacity (prevention of new sensitizations and deteriorations of disease severity) have not yet been established.

Peptide-based immunotherapy aims to induce peripheral T cell tolerance (anergy) without cross-linking IgE. This would avoid the problem of anaphylaxis potentially associated with traditional whole antigen-based immunotherapy. On the other hand, one limit is its specificity, i.e., the treatment is restricted to a specific antigen or a specific antigenic sequence of the whole antigen that contains multiple antigenic epitopes.

An exemplary anti-IgE therapy is a treatment with a “humanized” mouse monoclonal antibody that blocks IgE and the subsequent activation of its receptor FcεRI. Studies have documented the efficiency for anti-IgE to reduce IgE plasma levels, antigen-induced basophils activation, and the levels of FcεRI expression on basophils. Clinical studies indicated that the capacity of anti-IgE therapy to reduce asthma symptoms is weak.

DNA vaccines are based on the potential of immunostimulatory DNA sequences containing a CpG motif to inhibit the Th2 immune response. This treatment was found to reduce airway hyper-responsiveness in animal models of allergic inflammation. Ongoing clinical studies with immunostimulatory DNA and immunostimulatory DNA conjugated to protein allergens may determine the safety and efficacy of this immunomodulatory approach in the treatment of allergic disease.

The compositions of the present invention, namely, neurokinin-1 (NK-1) receptor antagonists in combination with an inhibitor of metachromatic cell activation, such as an anti-inflammatory agent, an immunosuppressor, or a kinase inhibitor, or a combination thereof, provide significant advantages over previously used compositions in reducing the side effect of the therapy as well as its cost.

Among the NK-1 receptor antagonists contemplated in this invention, some have been tested and validated for the treatment of human diseases, disorders or conditions related to the activation of the NK-1 receptor. Furthermore, combination of a selective neurokinin-1 receptor antagonist with an inhibitor of metachromatic cell activation, such as an anti-inflammatory agent (e.g., glucocorticoids) allows for reducing the concentrations of both molecules with a similar inhibitory effect on metachromatic cells activation. Exemplary advantages of the use of a NK-1 receptor antagonist in combination with an anti-inflammatory agent (e.g., glucocorticoids) include:

(1) A very potent blockage of IgE-related and non-antigenic related activation of metachromatic cells;

(2) No necessity for an immune treatment (sensitization or desensitization), thus avoiding side-effects and undesirable effects due to therapies based on immune system activation (e.g., fever, etc.);

(3) No potential problems arising from the use of some of the more well-established treatments (e.g., antihistamines, mast cell stabilizers, etc.) causing a sedative effect, and so causing a decrease in subject performance, alertness, and cognitive function; and

(4) Absence of depressive disorder that is often observed in patients with allergic diseases as reported in the asthmatic population. The use of an NK-1 receptor antagonist in combination with an anti-inflammatory agent (e.g., a glucocorticoid) should also have beneficial effect on the mental health of the asthmatic subjects because NK-1 receptor antagonists are efficient drugs to treat human depression.

Other features and advantages of the invention will be apparent from the following Detailed Description, the Drawings, and the Claims.

### BRIEF DESCRIPTION OF THE DRAWINGS

FIG. 1 is a graph showing the dose-response results of secretion of β-hexosaminidase, a marker of basophil activation and degranulation, in rat RBL-2H3 basophilic cells passively sensitized with mouse anti-IgE to dinitrophenyl conjugated to bovine serum albumin (DNP-BSA) and stimulated with DNP-BSA (1 to 40 ng/ml) for 30 minutes.

FIG. 2 is a graph showing the dose-response result of the potent and selective neurokinin-1 receptor antagonist L-703,606 oxalate salt to determine the amount required for inhibition of β-hexosaminidase release in IgE-sensitized RBL-2H3 cell line in response to low antigenic stimulation (DNP-BSA 5 ng/ml) and high antigenic stimulation (DNP-BSA 40 ng/ml).

FIG. 3 is a graph showing that inhibition of β-hexosaminidase release by the potent and selective neurokinin-1 receptor antagonist L-703,606 oxalate salt in IgE-sensitized RBL-2H3 cell line stimulated with DNP-BSA is not attributable to cell death.

FIG. 4 is a graph showing the result of the amount required for the potent and selective neurokinin-1 receptor antagonist to inhibit histamine release in IgE-sensitized RBL-2H3 cell line in response to a concentration of antigen (DNP-BSA 40 ng/ml) inducing maximal cell degranulation.

FIG. 5 is a graph showing the result of the amount required for the potent and selective neurokinin-1 receptor antagonist L-733,060 hydrochloride to inhibit βhexosaminidase release in IgE-sensitized RBL-2H3 cell line in response to a concentration of antigen (DNP-BSA 5 ng/ml) inducing 50% of maximal cell degranulation, and a concentration of antigen (DNP-BSA 40 ng/ml) inducing maximal cell degranulation.

FIG. 6 is a graph showing the result of the amount required for the potent and selective neurokinin-1 receptor antagonist WIN 51,708 to inhibit β-hexosaminidase release in IgE-sensitized RBL-2H3 cell line in response to a concentration of antigen (DNP-BSA 5 ng/ml) inducing 50% of maximal cell degranulation, and a concentration of antigen (DNP-BSA 40 ng/ml) inducing maximal cell degranulation.

FIG. 7 is a graph showing the result of the amount required for the potent and selective neurokinin-1 receptor antagonist RP 67580 to inhibit β-hexosaminidase release in IgE-sensitized RBL-2H3 cell line in response to a concentration of antigen (DNP-BSA 5 ng/ml) inducing 50% of maximal cell degranulation, and a concentration of antigen (DNP-BSA 40 ng/ml) inducing maximal cell degranulation.

FIG. 8 is a graph showing the lack of inhibition of β-hexosaminidase release in IgE-sensitized RBL-2H3 cell line in response to a concentration of antigen (DNP-BSA 5 ng/ml) inducing 50% of maximal cell degranulation, and a concentration of antigen (DNP-BSA 40 ng/ml) inducing maximal cell degranulation by the potent neurokinin-1 receptor antagonist Antagonist D, indicating the selectivity of neurokinin-1 receptor antagonists to inhibit metachromatic cell activation.

FIG. 9 is a graph showing the lack of inhibition β-hexosaminidase release in IgE-sensitized RBL-2H3 cell line in response to a concentration of antigen (DNP-BSA 5 ng/ml) inducing 50% of maximal cell degranulation, and a concentration of antigen (DNP-BSA 40 ng/ml) inducing maximal cell degranulation by MDL 105,212 a potent and dual antagonist of both the neurokinin-1 and neurokinin-2 receptors, indicating the selectivity for neurokinin-1 receptor antagonists to block metachromatic cell activation.

FIG. 10 is a graph showing the concentration-response results of the secretion of β-hexosaminidase in RBL-2H3 cell line in response to stimulation with calcium ionophore for 60 minutes.

FIG. 11 is a graph showing the dose-response result of the potent and selective neurokinin-1 receptor antagonist L-703,606 oxalate salt to measure the amount required for inhibition of β-hexosaminidase release in RBL-2H3 cell line in response to low calcium ionophore stimulation (0.5 to 1 μM) and high calcium ionophore stimulation (2.5 to 5 μM), indicating that potent and selective neurokinin-1 receptor antagonists as exemplified with the L-703,606 oxalate salt also inhibit non-antigenic activation and degranulation of metachromatic cells.

FIG. 12 is a graph showing that inhibition of calcium ionophore-induced β-hexosaminidase release by a potent and selective neurokinin-1 receptor antagonist as exemplified with the L-703,606 oxalate salt in the RBL-2H3 cell line is unlikely to be attributable to inhibition of calcium ionophore-induced calcium signaling in the RBL-2H3 cell line.

FIG. 13 is a graph showing that basophils contain immunoreactive-like substance P as assessed using a competitive enzyme immunoassay (EIA) for the quantification of substance P.

FIG. 14 is a graph showing that the commercially available EIA kit developed to selectively detect substance P also detects other tachykinin-related peptides such as hemokinins and endokinins.

FIG. 15 is a graph showing the efficacy of the combination of a neurokinin-1 receptor antagonist (as exemplified by L-703,606) and a glucocorticoid (as exemplified by dexamethasone) to repress or reduce the concentration of antigen (40 ng/ml DNP-BSA) that induces maximal cell activation and degranulation.

FIG. 16 is a graph showing the efficacy of the combination of a neurokinin-1 receptor antagonist (as exemplified by L-703,606) and a glucocorticoid (as exemplified by dexamethasone) to repress or reduce the dose of antigen (5 ng/ml DNP-BSA) that induces 50% of the maximal cell degranulation.

FIG. 17 is a graph of the time-course of the combination of a neurokinin-1 receptor antagonist (as exemplified by L-703,606) and a glucocorticoid (as exemplified by dexamethasone) showing that this combination increases and accelerates glucocorticoid-based reduction of maximal cell degranulation induced by the antigen DNP-BSA at the concentration of 40 ng/ml.

FIG. 18 is a graph showing the efficacy of the combination of a neurokinin-1 receptor antagonist (as exemplified by L-733,060) and a glucocorticoid (as exemplified by dexamethasone) to repress or reduce the concentration of antigen (40 ng/ml DNP-BSA) that induces maximal cell activation and degranulation.

FIG. 19 is a graph showing the efficacy of the combination of a neurokinin-1 receptor antagonist (as exemplified by L-703,606) and a glucocorticoid (as exemplified by fluticasone) to repress or reduce the concentration of antigen (40 ng/ml DNP-BSA) that induces maximal cell activation and degranulation.

FIG. 20 is a graph showing the efficacy of the combination of a neurokinin-1 receptor antagonist (as exemplified by L-703,606) and a glucocorticoid (as exemplified by flunisolide) to repress or reduce the concentration of antigen (40 ng/ml DNP-BSA) that induces maximal cell activation and degranulation.

FIG. 21 is a graph showing that the combination of a neurokinin-1 receptor antagonist (as exemplified by L-703,606) and an immunosuppressor (as exemplified by Tacrolimus/FK506) increases the inhibitory effects of this agent on antigen-induced maximal cell activation and degranulation.

FIG. 22 is a graph showing that the combination of a neurokinin-1 receptor antagonist (as exemplified by L-703,606) and an immunosuppressor (as exemplified by cyclosporin A) increases the inhibitory effects of this agent on antigen-induced maximal cell activation and degranulation.

FIG. 23 is a graph showing that the combination of a neurokinin-1 receptor antagonist (as exemplified by L-703,606) and an immunosuppressor (as exemplified by FTY720) increases the inhibitory effects of this agent on antigen-induced maximal cell activation and degranulation.

FIG. 24 is a graph showing that the combination of a neurokinin-1 receptor antagonist (as exemplified by L-703,606) and a Syk kinase inhibitor (as exemplified by BAY 61-3606) increases the inhibitory effects of this agent on antigen-induced maximal cell activation and degranulation.

FIG. 25 is a graph showing that the combination of a neurokinin-1 receptor antagonist (as exemplified by L-703,606) and a Src family kinase inhibitor (as exemplified by PP1) increases the inhibitory effects of this agent on antigen-induced maximal cell activation and degranulation.

FIG. 26 is a graph showing that the combination of a neurokinin-1 receptor antagonist (as exemplified by L-703,606) and a phosphatidylinositol 3-kinase (PI3K) inhibitor (as exemplified by LY-294,002) increases the inhibitory effects of this agent on antigen-induced maximal cell activation and degranulation.

FIG. 27 is a graph showing that the combination of a neurokinin-1 receptor antagonist (as exemplified by L-703,606) and a p38 MAP kinase inhibitor (as exemplified by SB202190) increases the inhibitory effects of this agent on antigen-induced maximal cell activation and degranulation.

FIG. 28 is a graph showing that the combination of a neurokinin-1 receptor antagonist (as exemplified by L-703,606) and a mitogen-activated protein kinase kinase (MAPKK) inhibitor (as exemplified by PD98,059) increases the inhibitory effects of this agent on antigen-induced maximal cell activation and degranulation.

### DETAILED DESCRIPTION OF THE INVENTION

There is a need and a vast market for the development of new therapeutic strategies to block IgE-dependent and IgE-independent activation of metachromatic cells that avoid the side effects of current pharmacologic treatments, or the risk of systemic anaphylactic reactions of allergen-specific immunotherapy. Such treatment should be safe, inexpensive, easy to administer, effective, and preferably with a capacity to interfere with the immunologic regulation of the allergic inflammation. The compositions of the invention described herein fulfill this need.

The present invention refers to the use of substance P antagonists, particularly the invention refers to the use of neurokinin-1 receptor antagonists in combination with an inhibitor of metachromatic cell activation, such as an anti-inflammatory agent (e.g., a glucocorticoid), an immunosuppressors, or a kinase inhibitor or other for the treatment of a wide variety of diseases, disorders and conditions associated with metachromatic cell, such as mast cell and/or basophil, activation (e.g., allergy, urticaria, etc.).

Metachromatic cells (i.e., mast cells and basophils) are often involved in antigenic and non-antigenic inflammatory response. Allergy is one of most common manifestations of an antigenic inflammatory response (the allergen being the antigen). The terms allergic, allergenic and antigenic response or reaction are used according to their usual definitions, i.e., to describe the reaction due to immune responses where the antibody most often is immunoglobulin (Ig) E (IgE). Disease due to allergies includes urticaria (commonly known as hives), hay-fever, asthma, rhinitis, and atopic dermatitis. Prevention of allergic reaction in a mammal (e.g., a human) is therefore a research area of great importance. The specific IgE antibodies bind to the specific IgE receptors of high affinity (FcεRI) expressed mainly on metachromatic cells, including both mast cells and basophils. The polyclonal nature of this process results in bridging and clustering of the IgE receptors, and subsequently in cell activation of mast cells. This activation triggers the release of various preformed and neo-synthesized mediators involved in the early and the late phase reactions of the symptomatic phase of allergy. The early allergic response is dependent on the IgE-mediated release of metachromatic cell-derived mediators such as histamine and leukotrienes. Metachromatic cells are also involved in other non-allergic diseases such as non-allergic urticaria, metachromatic cell-related autoimmune disorders, transplant rejection, injury, and other metachromatic cell-related disorders.

Disease due to allergies provokes the infiltration of specific tissues or organs with inflammatory cells and this, together with the resulting structural changes, causes the clinical features of symptoms. Indeed, inflammation is the primary cause of the condition, is responsible for symptoms exacerbation, and is an important determinant of both current and future severity of the disease. Successful treatment of the underlying inflammatory process improves symptom profile and quality of life. In asthma, the drug therapy is dependent on the symptoms severity and includes, for example:
Intermittent use of β2 adrenergic receptor agonists
Intermittent use of β2 adrenergic receptor agonists and regular use of inhaled anti-inflammatory agents (cromoglycate or low-dose glucocorticoids)
Intermittent use of β2 adrenergic receptor agonists and regular use of high-dose glucocorticoids.
Inhaled high-dose glucocorticoids and regular bronchodilators and methylxanthines or leukotriene antagonists
One of the above treatment plus oral prednisolone

Other strategies for treating asthma are based on allergen-specific immunotherapy that aims to block the activation of IgE/FcεRI complex.

U.S. Pat. Nos. 5,916,910, 6,274,627, 6,337,069, 6,407,135, 6,564,152, and 6,710,086 describe NK-1 receptor antagonists.

The mammalian tachykinin system currently includes three neuropeptides, namely substance P (SP), neurokinin (NK) A, and NKB, and three corresponding receptors NK-1, NK-2 and NK-3 which are members of the transmembrane G-protein-coupled receptor superfamily (Regoli et al., “Receptors and antagonists for substance P and related peptides,” Pharmacol Rev. 46:551-599, 1994; Moriarty et al., “Human colonic anti-secretory activity of the potent NK(1) antagonist, SR140333: assessment of potential anti-diarrhoeal activity in food allergy and inflammatory bowel disease,” Br. J. Pharmacol. 133(8):1346-1354, 2001; Wahlestedt, “Reward for persistence in substance P research,” Science 281:1624-1625, 1998). Tachykinins are autacoids, meaning that these neuropeptides have to be released in the vicinity of the target. The target is defined as the cell bearing NK-1 receptors or SP binding-proteins that will be activated when SP or other tachykinin-related peptides bind to the NK-1 receptor or SP binding-proteins. Tachykinins, in particular SP, are involved in several physiological and pathological processes including pain transmission, depression, emesis, neurogenic inflammation, allergy, and immunomodulation (Regoli et al., “Receptors and antagonists for substance P and related peptides,” Pharmacol Rev. 46:551-599, 1994; Moriarty et al., “Human colonic anti-secretory activity of the potent NK(1) antagonist, SR140333: assessment of potential anti-diarrhoeal activity in food allergy and inflammatory bowel disease,” Br. J. Pharmacol. 133(8):1346-1354, 2001; Wahlestedt, “Reward for persistence in substance P research,” Science 281:1624-1625, 1998; Bozic et al., “Neurogenic amplification of immune complex inflammation,” Science 273:1722-1725, 1996; Cao et al., “Primary afferent tachykinins are required to experience moderate to intense pain,” Nature 392:390-394, 1998). Tachykinins are released from c-sensitive fibers nerves and non-neuronal cells (Maghni et al., “Airway smooth muscle cells express functional NK-1 receptors and the nerve-derived preprotachykinin-A gene: Regulation by passive sensitization,” Am. J. Resp. Cell Mol. Biol. 28:103-110, 2003; Meloche et al., “Role of tachykinins in CD4+ T cells apoptosis: Determination of neurokinin-1 receptor and substance P expression in Jurkat T cells,” Immunology, Supplement, 479-483, 2004; Ouaked et al., “Evidence of autocrine tachykinergic regulation of basophil function,” Immunology, Supplement, 293-297, 2004).

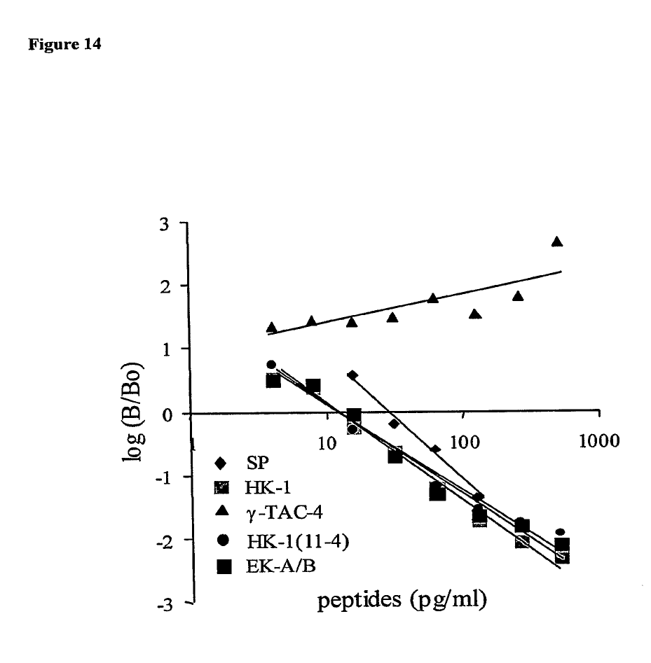

Hemokinins and endokinins are recently discovered tachykinin-related peptides that exert their actions solely and specifically through interactions with the NK-1 receptor (Page et al., “Characterization of the endokinins: human tachykinins with cardiovascular activity,” Proc. Natl. Acad. Sci. USA 100:6245-6250, 2003). Endokinins are expressed in peripheral tissues and organs (Page et al., “Characterization of the endokinins: human tachykinins with cardiovascular activity,” Proc. Natl. Acad. Sci. USA 100:6245-6250, 2003). The C-terminal immunogenic sequence of substance P is similar to the bioactive endokinins, namely endokinin-A and endokinin-B as well as mammalian hemokinin and the bioactive hemokinin 1-4 fragment. We (see, e.g., FIG. 14) and others (Page, “Hemokinins and endokinins,” Cell Mol. Life. Sci. 61:1652-1663, 2004) have shown that endokinins and hemokinins possess a strong cross-reactivity with a specific substance P antibody. Therefore, it is likely that the immuno-reactivity detected as substance P in biological samples and cell extracts would also correspond to the detection of hemokinins and/or endokinins. Furthermore, it is likely that in cells containing immuno-reactive substance P, the inhibition of cell activation by neurokinin-1 receptor antagonist may be also attributable of blocking autocrine action of endokinins and/or hemokinins. Therefore, the use of NK-1 receptor antagonists aims at further blocking the activation of metachromatic cells by hemokinins and/or endokinins, as well as other presently unknown molecules with similar or different chemical structures that act on the NK-1 receptor.

NK-1 receptor antagonists that may be included in the compositions of the present invention include the antagonists set forth in Table 1 below.

TABLE 1
Chemical and biochemical characteristics of the neurokinin-1 receptor
antagonists used

| Antagonist name | Chemical structure | | Structure | Affinity |
|---|---|---|---|---|
| RP 67580 | (3aR,7aR)- Octahydro-2-[1- imino-2-(2- methoxyphenyl) ethyl]-7,7-diphenyl- 4H-isoindol | 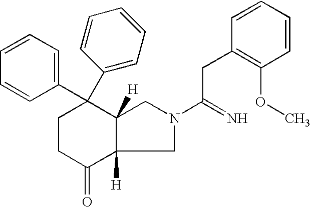 | Perhydrois oindolone | IC₅₀: 10 nM (rat brain) Ki: 4.2 (rat brain) pA2: 7.16 (guinea pig ileum) |
| WIN 51078 | 17-β-Hydroxy-17-α- ethynyl-5-α- androstano[3,2- b]pyrimido[1,2- a]benzimidazole | 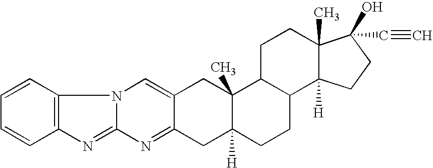 | Hetero steroid | IC₅₀: 50 nM (rat brain) |
| L-733,060 | ((2S,3S)-3-[[3,5-bis (Trifluoromethyl) phenyl]methoxy]-2- phenylpiperidine hydrochloride | 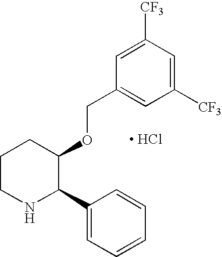 | Piperidine benzylether | IC₅₀: 1 nM (CHO cells expressing NK-1 receptors) |
| L-703,606 | cis-2- (Diphenylmethel)-N- ([2-iodophenyl] methyl)-1-azabicyclo (2.2.2) octan-3- amine | 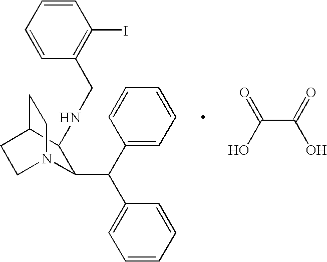 | Piperidine benzylether | IC₅₀: 2 nM (CHO cells expressing NK-1 receptors) Kd: 0.04 +/− 0.03 (65% sites) 1.5 +/− 0.7 nM (35% sites) |
| MDL 105,212 Dual NK1 and NK2 receptors | R)-1-[2-[3-(3,4- dichlorophenyl)-1- (3,4,5- trimethoxybenzoyl)- pyrrolidin-3-yl]- ethyl]-4- phenylpiperidine-4- carboxamide, hydrochloride- | 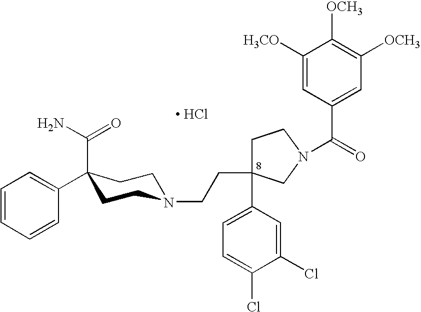 | Piperidine | IC₅₀: 3.11 nM |
| Antagonist D | [D-Arg¹, D-Phe⁵, D- | D-Arg-Pro-Lys-Pro-D-Phe- | — | Non-selective |
| | Trp^{7,9}, Leu¹¹] | Gln-D-Trp-Phe-D-Trp- | | NK-1 receptor |
| | substance P | Leu-Leu-NH₂ | | |

MDL-105212 is also described in Kudlacz et al. (J. Pharmacol. Exp. Ther. 277:840-851, 1996), and RP67580 is also described in Garret et al. (Proc. Natl. Acad. Sci. USA 88:10208-10212, 1991).

Aprepitant (MK-869 or L-754030; marketed by Merck & Co. under the name EMEND®) is a particularly desirable NK-1 receptor antagonist having the chemical formula C₂₃H₂₁F₇N₄O₃ and the structural formula:

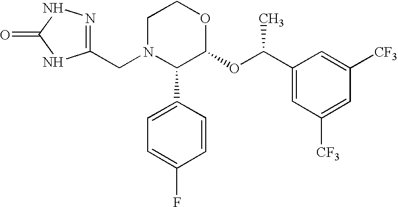

Chemically described as: 5-[[(2R,3S)-2-[(1R)-1-[3,5-bis(trifluoromethyl)phenyl]ethoxy]-3-(4-fluorophenyl)-4-morpholinyl]methyl]-1,2-dihydro-3H-1,2,4-triazol-3-one. The compound is also described in Kramer et al. (Science 281:1640-1645, 1998) and Rupniak and Kramer (Trends Pharmacol. Sci. 20:1-12, 1999).

Moreover, useful NK-1 receptor antagonists include those described in Megens et al. (The Journal of Pharmacology and Experimental Therapeutics 302:696-709, 2002) such as (2R-trans)-4-[1-[3,5-bis(Trifluoromethyl)benzoyl]-2-(phenylmethyl)-4-piperidinyl]-N-(2,6-dimethylphenyl)-1-acetamide(S)-Hydroxybutanedioate (R116301). Additional desirable NK-1 receptor antagonists include CGP49823 (Vassout et al., Neuropeptides 26(Suppl 1):38, 1994), CP-96345 (Snider et al., Proc. Natl. Acad. Sci. USA 88:10042-10044, 1991), CP-99994 (Piedimonte et al., J. Pharmacol. Exp. Ther. 266:270-273, 1993), GR-203040 (Ward et al., J. Med. Chem. 38:4985-4992, 1995), MDL-103392 (racemate of the active enantiomer MDL-105212), L-760735 (McAllister et al., Soc. Neurosci Abstr. 25 (Part 2) 733:11, 1999 (Abstract)), SDZ-NKT-343 (Walpole et al., J. Med. Chem. 41:3159-3173, 1998), and nolpitanitium (SR-140333; Edmonds-Alt et al., Eur. J. Pharmacol. 250:403-413, 1993; structure provided below).

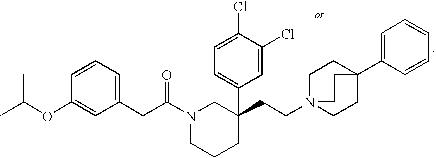

Other NK-1 receptor antagonists include the 1-aryl-2-acylamino-ethane compounds described in U.S. Pat. No. 5,929,067, LY686017 (Eli Lilly & Co.), 823296 (GlaxoSmithKline), H1/NK1 Dual Antagonists (Inflazyme Pharmaceuticals Ltd.), MPC-4505 (Myriad Genetics Inc.), CP-122721 (Pfizer Inc.), CJ-1 2,255 (Pfizer Inc.), SSR 240600 (Sanofi-Aventis), TA-5538 (Tanabe Seiyaku Co.), E-6006 (5-(alpha-[2-(dimethylamino)ethoxy]-2-thienylmethyl)-1-methyl-1H-pyrazole; structure provided below)

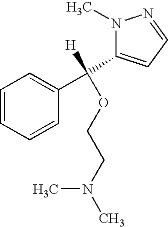

Vestipitant (2S)—N-{(1R)-1-[3,5-bis(trifluoromethyl)phenyl]ethyl}-2-(4-fluoro-2-methylphenyl)-N-methylpiperazine-1-carboxamide; structure provided below)

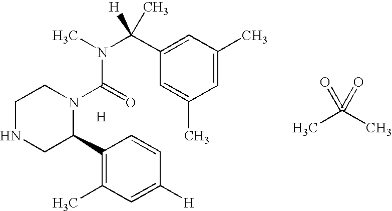

Netupitant (2-(3,5-Bis.trifluoromethyl-phenyl)-N-methyl-N-[6-(4-methyl-piperazin-1-yl)-4-0-tolyl-pyridin-3-yl]-isobutyramide; C₃₀H₃₂F₆N₄O; structure provided below)

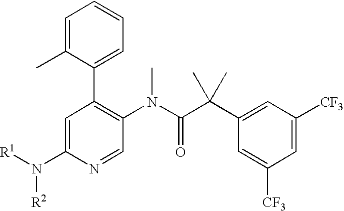

| | | [symbol] |
|---|---|---|
| Compound | NR¹R² | [symbol] |
| 21 [symbol] | 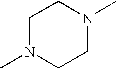 | 0.01 |
| 20 [symbol] | 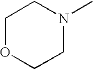 | 1.0 |

[symbol]

indicates data missing or illegible when filed

and Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide; structure provided below.)

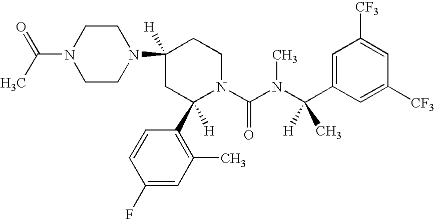

The compositions of the present invention are useful in treating syndromes and diseases that involve cells expressing a protein that binds IgE as exemplified by metachromatic cells activation and degranulation. In particular, the compositions of the present invention can be used to treat sensitivity to multiple irritants, non-antigenic stimuli, and antigenic stimuli in human subjects, and also animals, such as other vertebrates, including mammals, large and small, including wild and domesticated for veterinary purposes. Non-limiting examples of target diseases are allergy, urticaria, rhinitis, and asthma The invention can be also used to treat diseases related to the activation and the degranulation of metachromatic cells, including autoimmune diseases such as diabetes mellitus; multiple sclerosis; premature ovarian failure; scleroderma, Sjogren's disease; alopecia (baldness); polyglandular failure; Grave's disease; hypothyroidism; polymyosititis; Chron's disease; inflammatory bowel disease; autoimmune hepatitis; hypopituitarism; myocardititis; Addison's disease; autoimmune skin diseases; uveititis; pernicious anemia; hypoparathyroidism; rheumatoid arthritis; acne vulgaris; acute respiratory distress syndrome; allergic intraocular inflammatory diseases, ANCA-associated small-vessel vasculitis; ankylosing spondylitis; atopic dermatitis; autoimmune hemolytic anemia; Behcet's disease; Bell's palsy; bullous pemphigoid; cerebral ischaemia; cirrhosis; Cogan's syndrome; contact dermatitis; Cushing's syndrome; dermatomyositis; discoid lupus erythematosus; lupus nephritis; eosinophilic fasciitis; erythema nodosum; exfoliative dermatitis; focal glomerulosclerosis; focal segmental glomerulosclerosis; segmental glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic thrombocytopenic purpura; immune thrombocytopenic purpura inflammatory bowel or gastrointestinal disorders, inflammatory dermatoses; lichen planus; lymphomatous tracheobronchitis; macular edema; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; septic shock syndrome; shoulder tendinitis or bursitis; Still's disease; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; vasculitis; and Wegener's granulomatosis.

The compositions of the present invention which include an inhibitor of NK-1 receptor activity (i.e., a NK-1 receptor antagonist) and an inhibitor of metachromatic cell activation, such as an anti-inflammatory agent (exemplified by a glucocorticoid) block non-antigenic and antigen-induced basophil/mast cell activation and the subsequent release of preformed and neo-synthesized inflammatory mediators.

The combinations of the invention inhibit the activity of cell membrane receptors (e.g., NK-1 and FcεRI receptors) and other membrane-bound receptors and/or other membrane-bound entities, as well as intracellular receptors and/or entities which are constitutively expressed, up-regulated or induced in inflammatory conditions.

The inhibition of an NK-1 receptor may be achieved by direct and indirect means. Indirect means include capturing the receptor agonists (e.g., tachykinins such as Substance P, and related molecules such as hemokinins and endokinins) by using ligands like monoclonal antibodies or any molecules having an affinity for the agonists and hindering the natural liaison between the agonists and the receptor. Inhibition also includes methods aimed at inducing the degradation of Substance P and related molecules such as hemokinins and endokinins into biological inactive substances, which would reduce the amount of active agonists. Further, indirect inhibition may involve inhibition of the biological activity of a component of an NK-1 receptor signaling pathway. Direct means include ligands that bind directly to the receptor or Substance P binding-protein and reduce or inhibit its activity. Monoclonal antibodies, and competitive and non-competitive pharmaceutical antagonists are examples of a direct inhibition.

The present invention discloses that NK-1 receptor antagonists block antigenic and non-antigenic stimuli-induced metachromatic cells activation. This finding supports a role for endogenous tachykinins and tachykinin-related peptides in the process of metachromatic cells activation. The present invention relates to the use, method of use and compositions of NK-1 receptor antagonists in combination with an inhibitor or metachromatic cell activation capable of complementing their action.

Glucocorticoids, immunosuppressors, and kinase inhibitors were specifically tested and, together with NK-1 receptor antagonists, act by blocking autocrine action of endogenous tachykinins and tachykinin-related peptides more than each component alone.

As detailed below, we have shown that pre-treatment with selective neurokinin-1 receptor antagonists in combination with an anti-inflammatory compound, immunosuppressor agent, or kinase inhibitor has a synergistic effect that surpasses the effect of each compound used alone in repressing or reducing metachromatic cell activation and degranulation. The combination of neurokinin-1 receptor antagonists with anti-inflammatory agent, immunosuppressor agent, or kinase inhibitors is therefore useful in the treatment of diseases, syndromes, and disorders related to metachromatic cell activation and degranulation.

### Pharmaceutical Compositions

The compounds and combination of compounds of the present invention are useful in the treatment of conditions or diseases associated with metachromatic cell activation. Generally, such treatments involve administering to a subject in need thereof an effective amount of a compound containing an NK-1 receptor inhibitor (e.g., L-703,606, L-733,060, WIN-51,708, RP67580, or MDL-105,212) an a compound that inhibits metachromatic cell activation, such as a glucocorticoid (e.g., dexamethasone, fluticasone, flunisolide, or mometasone), an immunosuppressor (e.g., Tacrolimus/FK506, Cyclosporin A, or myriocin derivatives) or a kinase inhibitor (e.g., syk kinase inhibitor BAY 61-3606 src kinase family inhibitor PP1, PI3K inhibitor LY-294,002, p38 MAP kinase inhibitor SB202190, or MAPKK inhibitor PD98,059), or a combination of such compounds, and a suitable pharmaceutical carrier. The subject desirably is a mammal (e.g., a human). The disorder desirably is an allergy, urticaria, rhinitis, or asthma.

The pharmaceutical compositions can be in a variety of forms including oral dosage forms, topic creams, suppository, nasal spray and inhaler, as well as injectable and infusible solutions. Methods for preparing pharmaceutical composition are well known in the art.

Compositions within the scope of the present invention desirably contain the active agent (e.g., a combination of an NK-1 receptor inhibitor and an inhibitor of metachromatic cell activation described herein) in an amount effective to achieve the desired therapeutic effect while avoiding adverse side effects. Pharmaceutically acceptable preparations and salts of the active agent are within the scope of the present invention and are well known in the art. For the administration of a combination of compounds of the invention, the amount administered desirably is chosen so as to avoid adverse side effects. The amount of the therapeutic or pharmaceutical composition which is effective in the treatment of a particular disease, disorder or condition depends on the nature and severity of the disease, the target site of action, the patient's weight, special diets being followed by the patient, concurrent medications being used, the administration route and other factors that are recognized by those skilled in the art. The dosage can be adapted by the clinician in accordance with conventional factors such as the extent of the disease and different parameters from the patient. Typically, 0.001 to 100 mg/kg/day is administered to the subject. Effective doses may be extrapolated from dose response curves derived from in vitro or animal model test systems. For example, in order to obtain an effective mg/kg dose for humans based on data generated from rat studies, the effective mg/kg dosage in a rat is divided by six.

Various delivery systems are known and can be used to administer the combinations of compounds described herein. The pharmaceutical composition of the present invention can be administered by any suitable route including, intravenous or intramuscular injection, intraventricular or intrathecal injection (for central nervous system administration), orally, topically, subcutaneously, subconjunctivally, or via intranasal, intradermal, sublingual, vaginal, rectal or epidural routes.

Other delivery system well known in the art can be used for delivery of the pharmaceutical compositions of the present invention, for example via aqueous solutions, encapsulation in nanoparticles, microparticles, or microcapsules.

The pharmaceutical compositions of the present invention can also be delivered in a controlled release system. For example, a polymeric material can be used (see, e.g., Smolen and Ball, Controlled Drug Bioavailability, Drug product design and performance, 1984, John Wiley & Sons; Ranade and Hollinger, Drug Delivery Systems, pharmacology and toxicology series, 2003, 2^{nd} edition, CRRC Press). Alternatively, a pump may be used (Saudek et al., N. Engl. J. Med. 321:574 (1989)).

The compounds of the present invention may also be coupled to a class of biodegradable polymers useful in achieving controlled release of the drug, for example, polylactic acid, polyorthoesters, cross-linked amphipathic block copolymers and hydrogels, polyhydroxy butyric acid, and polydihydropyrans.

The term carrier, in reference to a pharmaceutically acceptable carrier, refers to diluents, adjuvants, excipients or vehicles with which the compound or combination of compounds is administered. Such pharmaceutical carriers include sterile liquids such as water and oils including mineral oil, vegetable oil (e.g., peanut oil, soybean oil, sesame oil), animal oil or oil of synthetic origin. Aqueous glycerol and dextrose solutions as well as saline solutions may also be employed as liquid carriers of the pharmaceutical compositions of the present invention. The choice of the carrier depends on factors well recognized in the art, such as the nature of the peptide, peptide derivative or peptidomimetic, its solubility and other physiological properties as well as the target site of delivery and application. For example, carriers that can penetrate the blood brain barrier are used for treatment, prophylaxis or amelioration of symptoms of diseases or conditions (e.g. inflammation) in the central nervous system. Examples of suitable pharmaceutical carriers are described in Remington: The Science and Practice of Pharmacy by Alfonso R. Gennaro, 2003, 21^{th} edition, Mack Publishing Company.

Further pharmaceutically suitable materials that may be incorporated in pharmaceutical preparations of the present invention include absorption enhancers, pH regulators and buffers, osmolarity adjusters, preservatives, stabilizers, antioxidants, surfactants, thickeners, emollient, dispersing agents, flavoring agents, coloring agents, and wetting agents.

Examples of suitable pharmaceutical excipients include, water, glucose, sucrose, lactose, glycol, ethanol, glycerol monostearate, gelatin, starch flour (e.g., rice flour), chalk, sodium stearate, malt, sodium chloride, and the like. The pharmaceutical compositions of the present invention can take the form of solutions, capsules, tablets, creams, gels, powders sustained release formulations and the like.

The composition can be formulated as a suppository, with traditional binders and carriers such as triglycerides (see Remington: The Science and Practice of Pharmacy by Alfonso R. Gennaro, 2003, 21^{th} edition, Mack Publishing Company). Such compositions contain a therapeutically effective amount of the therapeutic composition, together with a suitable amount of carrier so as to provide the form for proper administration to the subject. The formulations are designed to suit the mode of administration and the target site of action (e.g., a particular organ or cell type).

The pharmaceutical compositions of the present invention can be formulated as neutral or salt forms. Pharmaceutically acceptable salts include those that form with free amino groups and those that react with free carboxyl groups. Non-toxic alkali metal, alkaline earth metal, and ammonium salts commonly used in the pharmaceutical industry include sodium, potassium, lithium, calcium, magnesium, barium, ammonium, and protamine zinc salts, which are prepared by methods well known in the art. Also included are non-toxic acid addition salts, which are generally prepared by reacting the compounds of the present invention with suitable organic or inorganic acid. Representative salts include the hydrobromide, hydrochloride, valerate, oxalate, oleate, laureate, borate, benzoate, sulfate, bisulfate, acetate, phosphate, tysolate, citrate, maleate, fumarate, tartrate, succinate, napsylate salts, and the like.

The compounds and combination of compounds of present invention may be administered alone or in combination with other active agents useful for the treatment, prophylaxis or amelioration of symptoms of a disease or condition involving activation of metachromatic cells. Thus, the compositions and methods of the present invention can be used in combination with other agents exhibiting the ability to modulate metachromatic cell activity or to reduce the symptoms of a disease associated with activation of metachromatic cells.

The invention is described herein below by reference to specific examples, embodiments and figures, the purpose of which is to illustrate the invention rather than to limit its scope. The following examples are not to be construed as limiting.

### Example 1

### Activation of Metachromatic Cells Involves NK-1 Receptors

The rat basophilic cell line RBL-2H3 is an art recognized model for studying the activation of metachromatic cells through FcεRI (Oliver et al., “Signal transduction and cellular response in RBL-2H3 mast cells,” Prog. Allergy 42:185-245, 1988). Recently, we have shown that RBL-2H3 cells express both SP and its receptor, the NK-1 receptor (Ouaked et al., Immunology, Suppl., 293:97, 2005). These finding support the existence of autocrine tachykinergic regulation of metachromatic cell function that involves SP and its receptor.

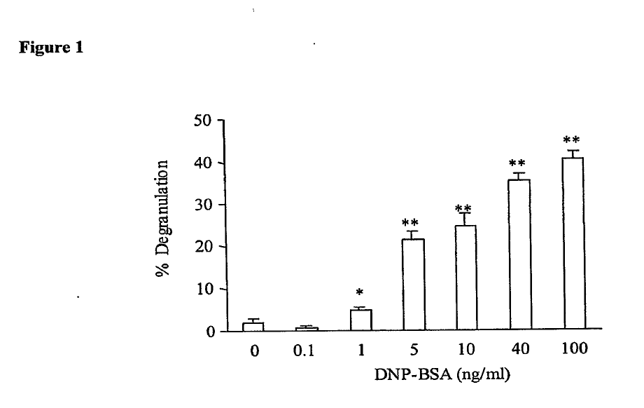

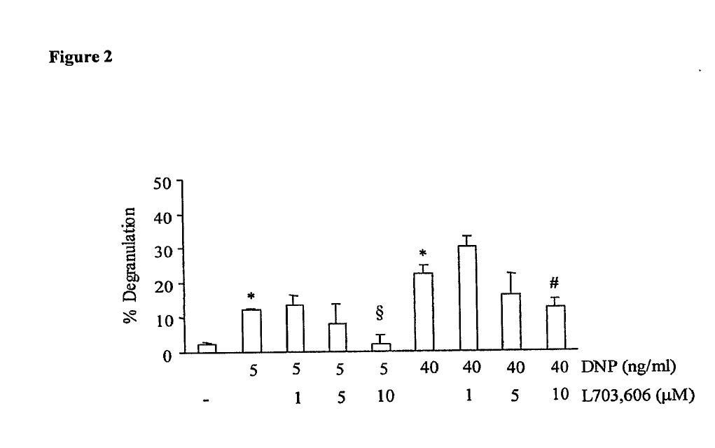

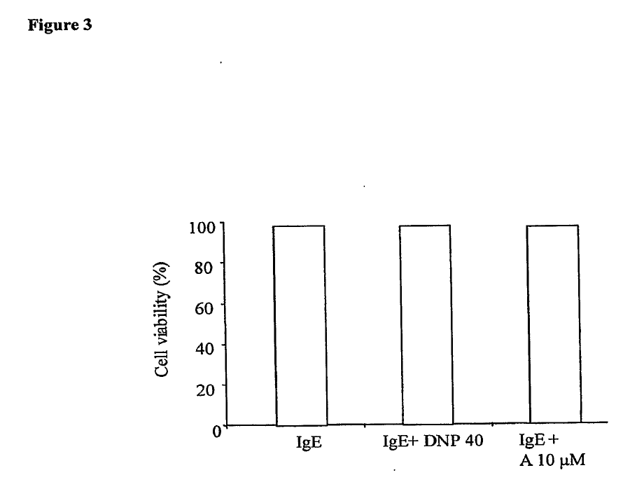

The below experiments used the rat basophilic cells RBL-2H3 commercially available from the American Type Culture Collection (ATCC; Manassas, Va.) under accession number CRL-2256. RBL-2H3 cells were passively sensitized with mouse IgE anti-dinitrophenyl conjugated to bovine serum albumin (DNP-BSA) monoclonal antibody for 22 hours, and then stimulated with DNP-BSA (antigen) to induce cell degranulation (FIG. 1). To address the issue of an autocrine tachykinergic regulation of metachromatic cell degranulation in response to antigenic activation, IgE-sensitized RBL-2H3 cells were pre-treated with a selective NK-1 receptor antagonist prior to antigen stimulation. The potency of L-703,606 (SigmaAldrich # L-119) (1 to 10 μM) to modulate antigen-induced β-hexosaminidase release was examined at concentrations of DNP-BSA that induce 50% of the maximal cells degranulation (5 ng/ml) or the maximal cell degranulation (40 ng/ml) (FIG. 2). The data indicate that L-703,606 causes a concentration-dependent inhibition of β-hexosaminidase release in RBL-2H3 cells, 10 μM being the most efficient inhibitory concentration (FIG. 2). Antigen-induced 50% of cell degranulation was completely abolished by L-703,606 (10 μM), and the maximal antigenic degranulation response was reduced by nearly 50% (FIG. 2B). L-703,606 did not change the basal release of β-hexosaminidase (data not shown), and did not alter cell viability as assessed by the trypan blue exclusion test (FIG. 3).

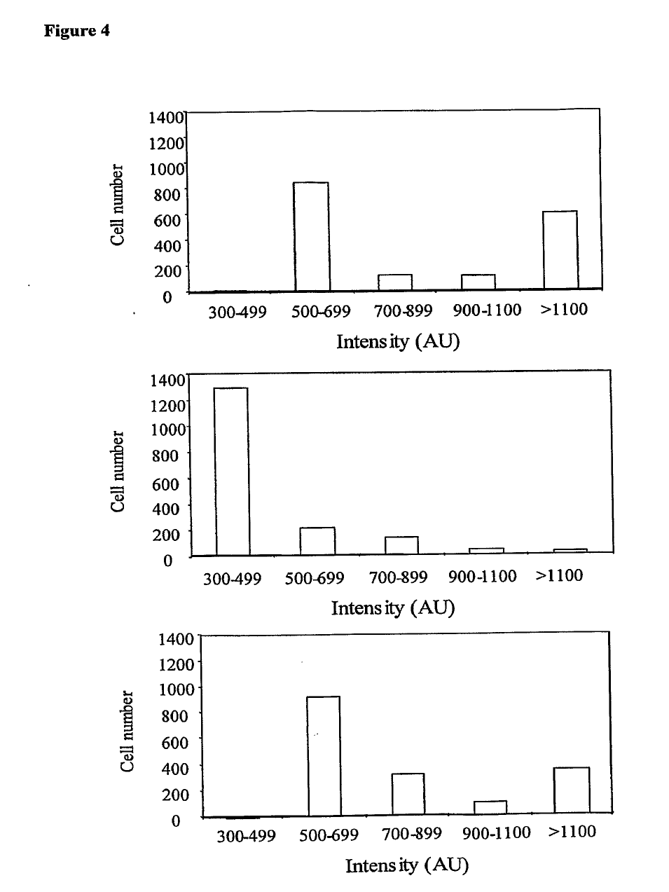

To further demonstrate that NK-1 receptors are involved in FCεRI clustering-induced metachromatic cell degranulation, changes in the intracellular pools of histamine were quantified. Data analysis using the macro built under the Image-Pro Plus platform enabled the representation of the frequency distribution of the maximal intensity for histamine immunoreactivity into RBL-2H3 cells. We arbitrarily divided basophils into six subgroups according to the range of maximal fluorescence intensity detected into the cells, e.g., 300 to 499 IAU (intensity arbitrary unit) or 900-1100 IAU. As shown in FIG. 4, histamine immunoreactivity was detected in IgE-sensitized RBL-2H3 cells (FIG. 4, upper panel), and the stimulation with DNP-BSA (40 ng/ml) depleted most cells of their histamine contents (FIG. 4, center panel). The depletion of histamine intracellular pools was mostly abolished following cells pretreatment with 10 μM of L-703,606 (FIG. 4, lower panel). Therefore, frequency distribution analyses confirmed the β-hexosaminidase data by showing that blocking NK-1 receptors inhibits FcεRI clustering-induced metachromatic cell degranulation.

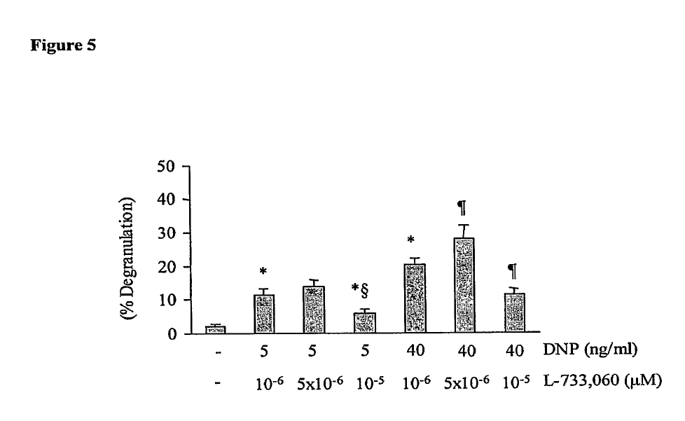

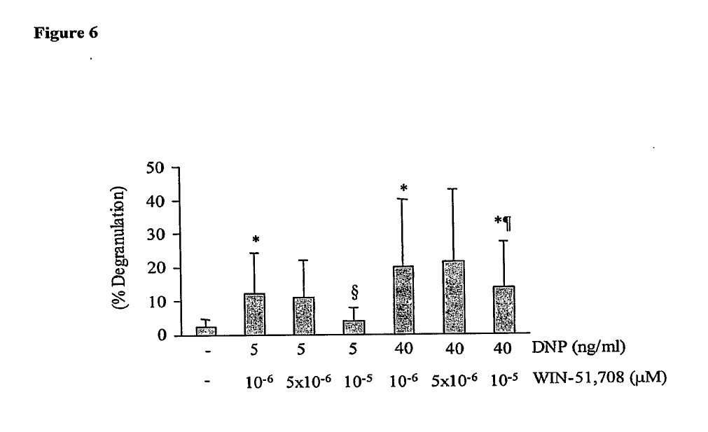

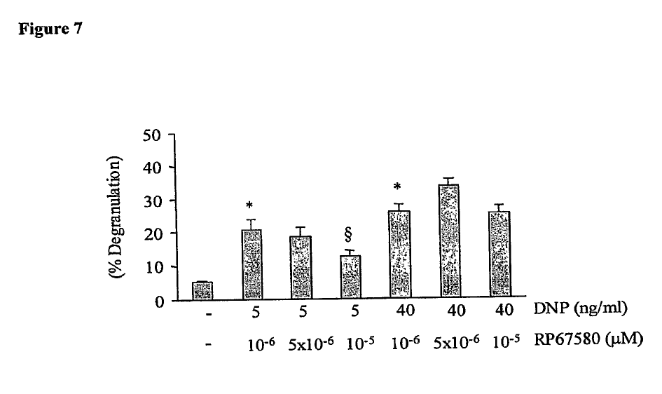

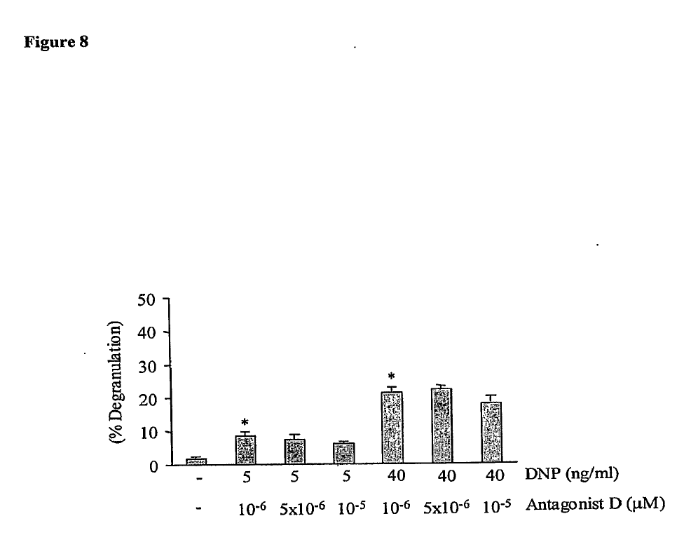

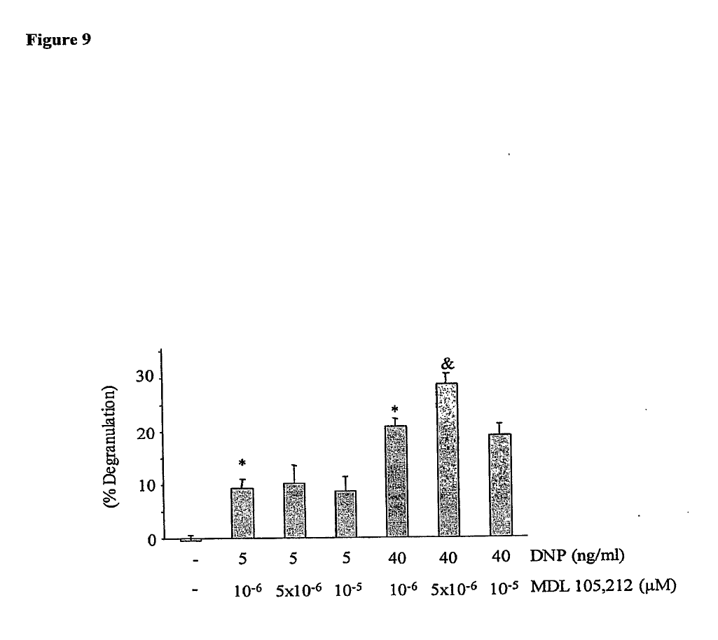

The efficiency of NK-1 receptor antagonists to reduce antigen-induced degranulation was further examined using three other selective NK-1 receptor antagonists, and one dual tachykinin receptors antagonist. Antigen-induced 50% of cells degranulation was completely abolished by L-733,060 (Tocris # 1145) (10 μM), and the maximal degranulation response was reduced by nearly 55% (FIG. 5). Similar levels of inhibition of antigen-induced basophils degranulation were found with the NK-1 receptor antagonist WIN-51,708 (SigmaAldrich #W-103) (10 μM) (FIG. 6), whereas RP67580 (Tocris #1635) (10 μM) decreased by nearly 50% the degranulation of metachromatic cell induced by 5 ng/ml DNP-BSA, but had no significant effect on the maximal antigenic degranulation of the cells (FIG. 7). Antagonist D (SigmaAldrich # S-3144), a peptidic NK-1 receptor antagonist that induces apoptotic cell death of small lung cancer cells, did not alter the antigenic degranulation of metachromatic cell (FIG. 8). RBL-2H3 cells were also pre-treated with the dual NK-1/NK-2 receptors antagonist, MDL-105,212 (A.G. Scientific, Inc. # M-1092). MDL-105,212 at both 1 μM and 10 μM had no effect on the antigen concentration leading to 50% of maximal metachromatic cell degranulation (FIG. 9). However, at the concentration of 1 μM, but not 10 μM, MDL-105,212 significantly increased cell degranulation induced by 40 ng/ml DNP-BSA FIG. 9). None of these antagonists showed an effect on the basal release of β-hexosaminidase (data not shown). Thus, pre-treatment with selective neurokinin-1 receptor antagonists represses or reduces antigen-induced metachromatic cell activation and degranulation.

### Example 2

### Non-Antigenic Stimulation Also Affects Activation of Metachromatic Cells by NK-1 Receptor Activation

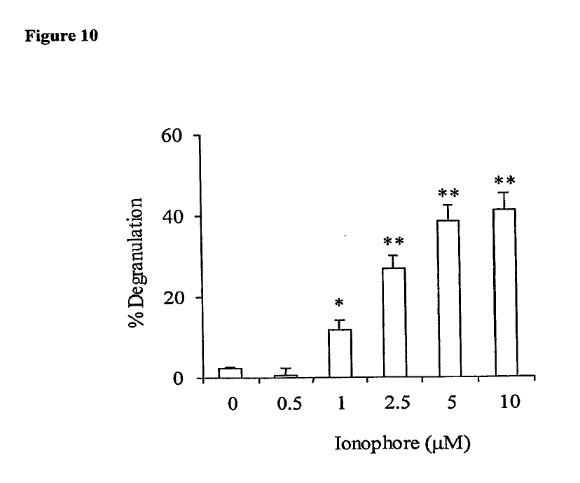

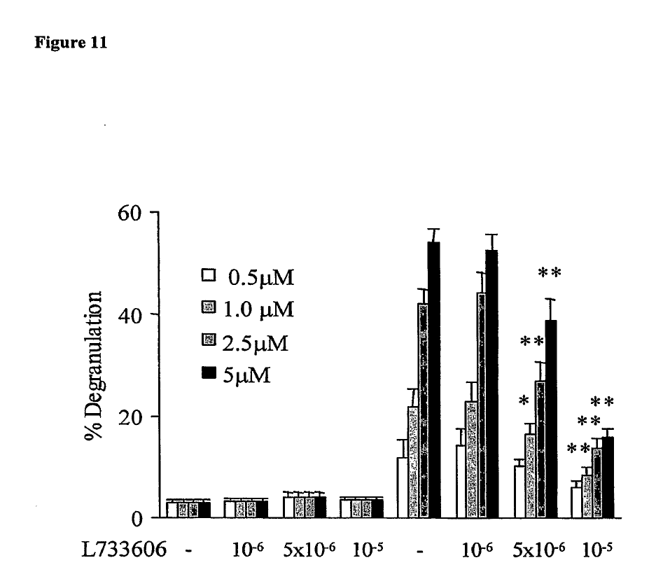

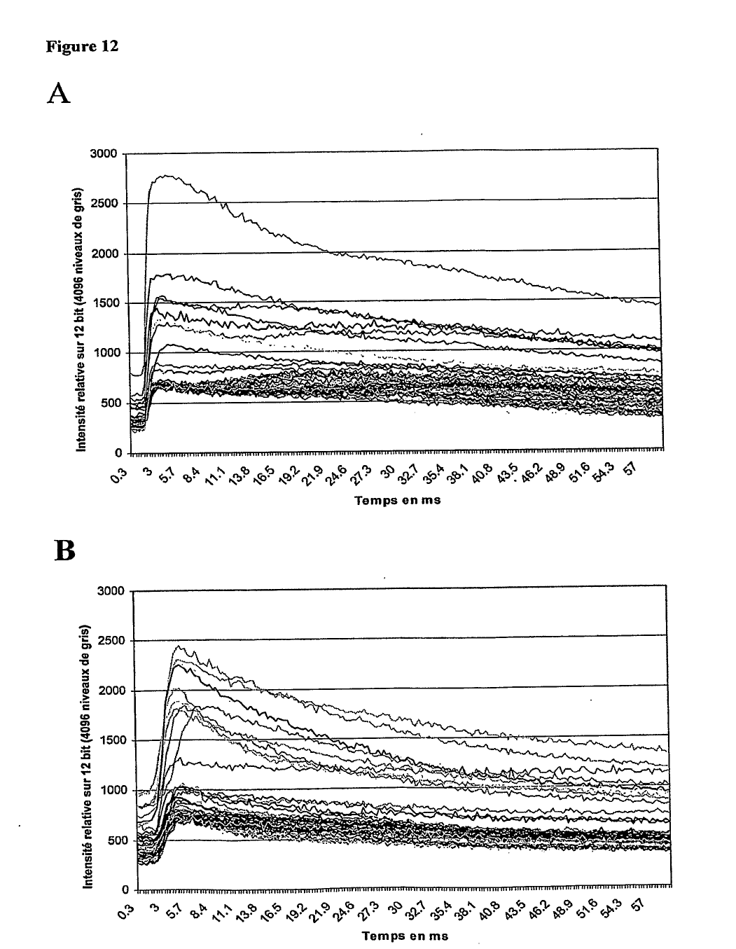

RBL-2H3 cells were stimulated with the calcium ionophore A23187 (SigmaAldrich #C7522) in a concentration-dependent manner to induce metachromatic cell degranulation FIG. 10). The data indicate that neurokinin-1 receptor antagonists as exemplified by L-703,606 inhibit, in a concentration-dependent manner, calcium ionophore-induced metachromatic cell activation and degranulation. The maximal inhibition is obtained at the concentration of 10 μM of L-703,606 (FIG. 11). To demonstrate that this inhibition is not attributable to direct interactions (e.g., chelation) between L-703,606 and the calcium ionophore, RBL-2H3 cells were pre-loaded with the calcium probe Fluo-3 AM (SigmaAldrich, # F6142), and intracellular changes of calcium in cells were determined in live cells by epifluorescence microscopy, and data analyzed using ImagePro Plus software (MediaCybernetics). The data indicate that inhibition of ionomycin-induced β-hexosaminidase release by the selective neurokinin-1 receptor antagonist L-703,606 oxalate salt in the RBL-2H3 cell line is unlikely to be attributable to inhibition of calcium ionophore-induced extracellular calcium signals in these cells (FIG. 12). Taken together, these data indicate that pre-treatment with selective neurokinin-1 receptor antagonists represses or reduces metachromatic cell activation and degranulation, and, thus, has applications in the treatment of diseases, syndromes, and disorders associated with non-IgE-dependent activation of metachromatic cells.

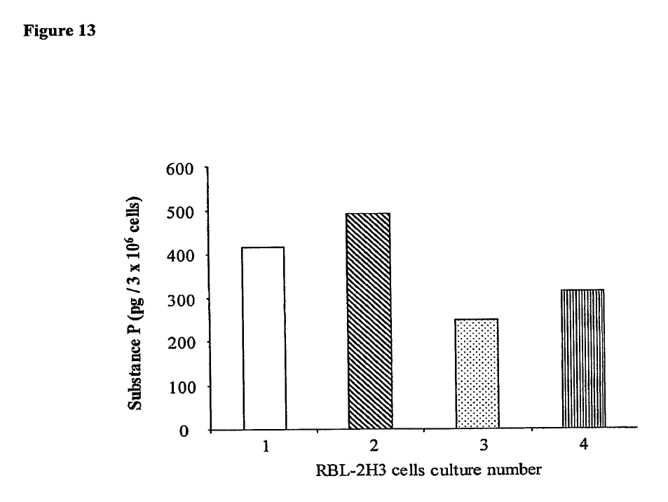

FIG. 13 indicates that metachromatic cells contain immunoreactivity for SP. The discovery of a third mammalian tachykinin gene, the preprotachykinin-C (PPT-C) gene or Tac4 gene (Page et al., “Characterization of the endokinins: human tachykinins with cardiovascular activity,” Proc. Natl. Acad. Sci. USA 100:6245-6250, 2003; Page, “Hemokinins and endokinins,” Cell Mol. Life. Sci. 61:1652-1663, 2004) encoding for endokinins (EKA/B) and hemokinins (HK-1 and HK-1 (Bozic et al., “Neurogenic amplification of immune complex inflammation,” Science 273:1722-1725, 1996; Cao et al., “Primary afferent tachykinins are required to experience moderate to intense pain,” Nature 392:390-394, 1998; Maghni et al., “Airway smooth muscle cells express functional NK-1 receptors and the nerve-derived preprotachykinin-A gene: Regulation by passive sensitization,” Am. J. Resp. Cell Mol. Biol. 28:103-110,2003; Meloche et al., “Role of tachykinins in CD4+ T cells apoptosis; Determination of neurokinin-1 receptor and substance P expression in Jurkat T cells,” Immunology, Supplement, 479-483, 2004; Ouaked et al., “Evidence of autocrine tachykinergic regulation of basophil function,” Immunology, Supplement, 293-297, 2004; Page et al., “Characterization of the endokinins: human tachykinins with cardiovascular activity,” Proc. Natl. Acad. Sci. USA 100:6245-6250, 2003; Page, “Hemokinins and endokinins,” Cell Mol. Life. Sci. 61:1652-1663, 2004; Oliver et al., “Signal transduction and cellular response in RBL-2H3 mast cells,” Prog. Allergy 42:185-245, 1988)), peptides that contain the tachykinin signature motif (FXGLM-NH₂; SEQ ID NO:2) has questioned whether assays are specific to SP detection and quantification. Our data indicate that the Cayman SP EIA detection kit displays strong cross-reactivity for EKA/B, HK-1 and its truncated version HK-1 (Bozic et al., “Neurogenic amplification of immune complex inflammation,” Science 273:1722-1725, 1996; Cao et al., “Primary afferent tachykinins are required to experience moderate to intense pain,” Nature 392:390-394, 1998; Maghni et al., “Airway smooth muscle cells express functional NK-1 receptors and the nerve-derived preprotachykinin-A gene: Regulation by passive sensitization,” Am. J. Resp. Cell Mol. Biol. 28:103-110, 2003; Meloche et al., “Role of tachykinins in CD4+ T cells apoptosis: Determination of neurokinin-1 receptor and substance P expression in Jurkat T cells,” Immunology, Supplement, 479-483, 2004; Ouaked et al., “Evidence of autocrine tachykinergic regulation of basophil function,” Immunology, Supplement, 293-297, 2004; Page et al., “Characterization of the endokinins: human tachykinins with cardiovascular activity,” Proc. Natl. Acad. Sci. USA 100:6245-6250, 2003; Page, “Hemokinins and endokinins,” Cell Mol. Life. Sci. 61:1652-1663, 2004; Oliver et al., “Signal transduction and cellular response in RBL-2H3 mast cells,” Prog. Allergy 42:185-245, 1988). No cross-reactivity was found for γ-TAC4 (32-50)-NH2 (FIG. 14). The SP and HK-1 tachykinins produced by metachromatic cells are equally potent agonists for the activation of the NK-1 receptor, and share common signaling pathways. Therefore, the inhibitory effect of neurokinin-1 receptor antagonists in IgE-dependent or IgE-independent activation of metachromatic cells may be attributable to blockage of SP, endokinins/hemokinins, or both.

### Example 3

### Combination of an NK-1 Receptor Antagonist and a Steroid

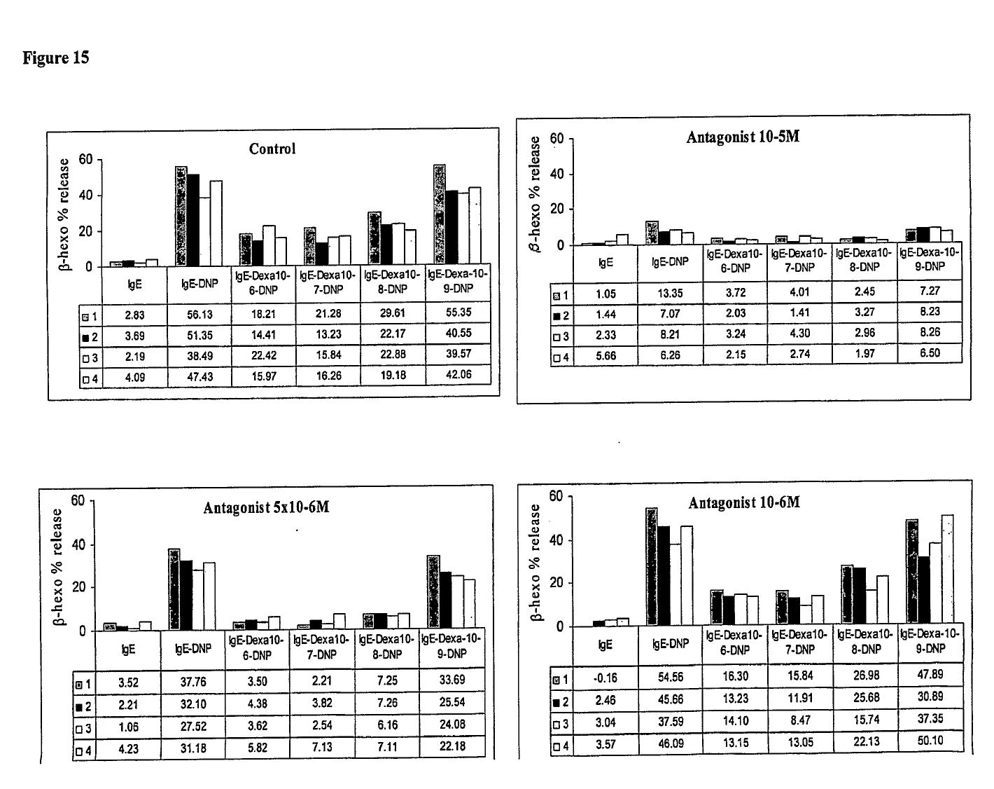

As noted above, neurokinin-1 receptor antagonists in combination with steroids are useful for the treatment of a wide variety of diseases, disorders, and conditions associated with metachromatic cell activation (e.g., allergy, urticaria, etc.). As an illustration of the efficiency of such a combination, our data indicate that the neurokinin-1 receptor antagonist L-703,606 (10 μM) in combination with the glucocorticoid dexamethasone (10⁻⁸ M) completely abolished the dose of antigen (40 ng/ml) that induces maximal cell degranulation, dexamethasone 10⁻⁸ M alone reduced the maximal degranulation by only 50% (FIG. 15). Furthermore, the data indicate that 5 μM L-703,606 has a weak effect to antigen-induced maximal degranulation; however the combination of 5 μM L-703,606 and Dexamethasone 10⁻⁸ M completely abolished antigen-induced maximal cell degranulation (FIG. 15).

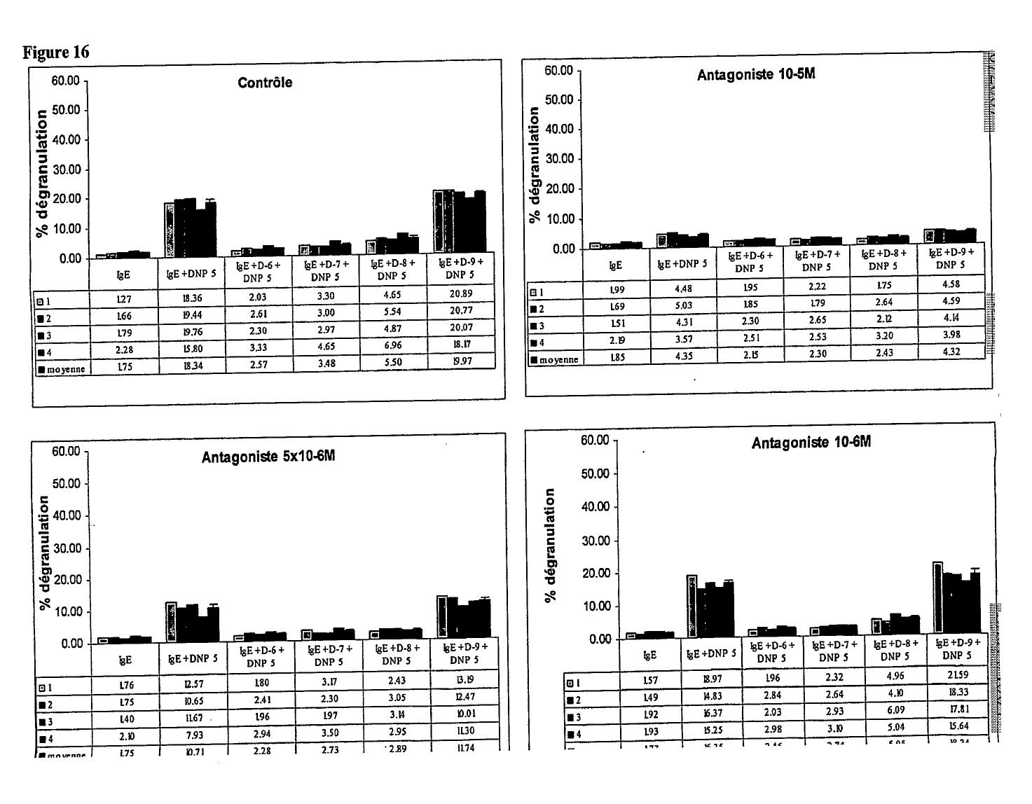

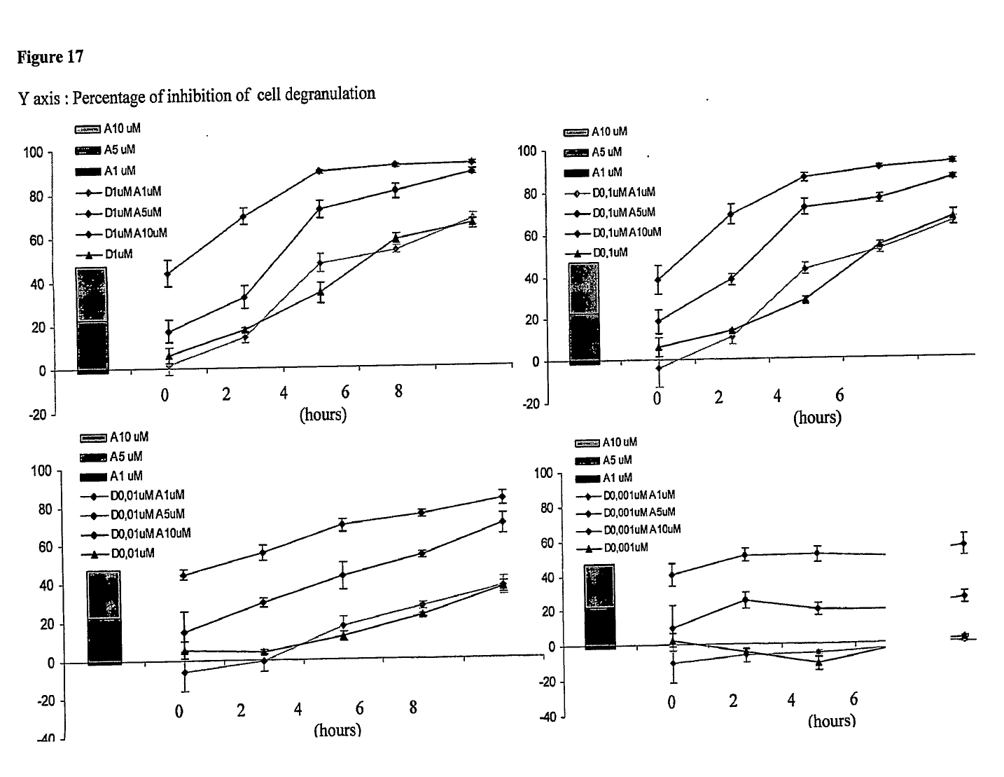

A study of the effect of this combination on the concentration of the antigen that induces 50% of the maximal degranulation indicated that 5 μM L-703,606 and Dexamethasone 10⁻⁸ M, or 1 μM L-703,606 and Dexamethasone 10⁻⁷ M, are as efficient as Dexamethasone 10⁻⁶ M alone in completely abolishing cell degranulation (FIG. 16). A time-course study of the combination of neurokinin-1 receptor antagonist L-703,606 and dexamethasone (FIG. 17) has further confirmed that (a) this combination increases the inhibitory effects of dexamethasone, and (b) enhances the efficiency of dexamethasone by lowering the concentration of the anti-inflammatory agent requires for similar inhibitory action, and also indicates that this combination accelerates the anti-inflammatory effects of dexamethasone.

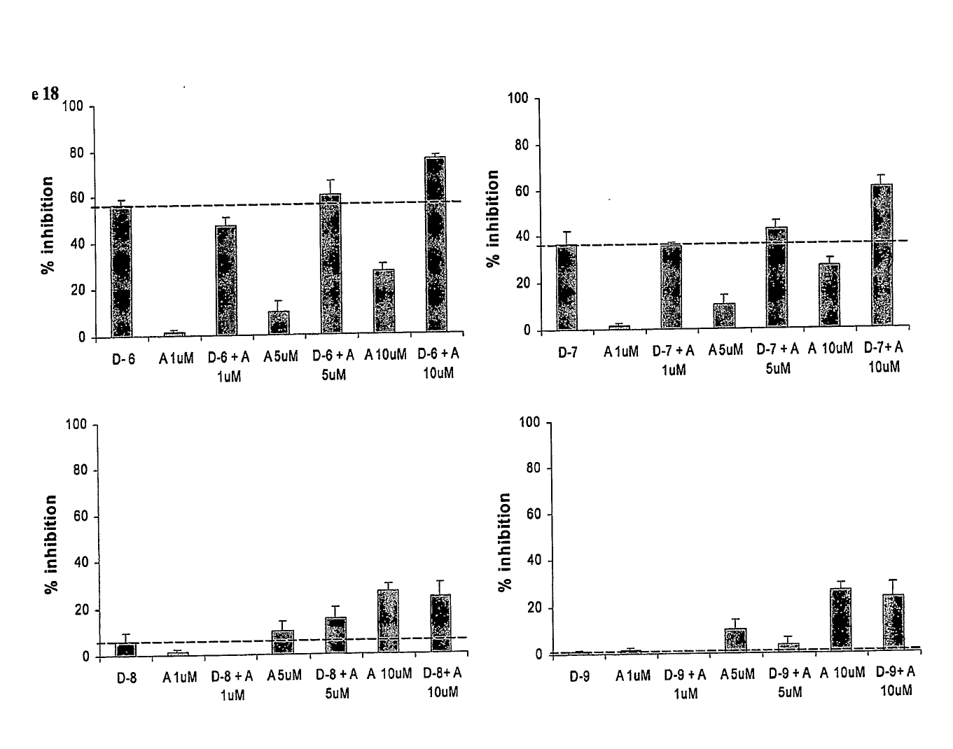

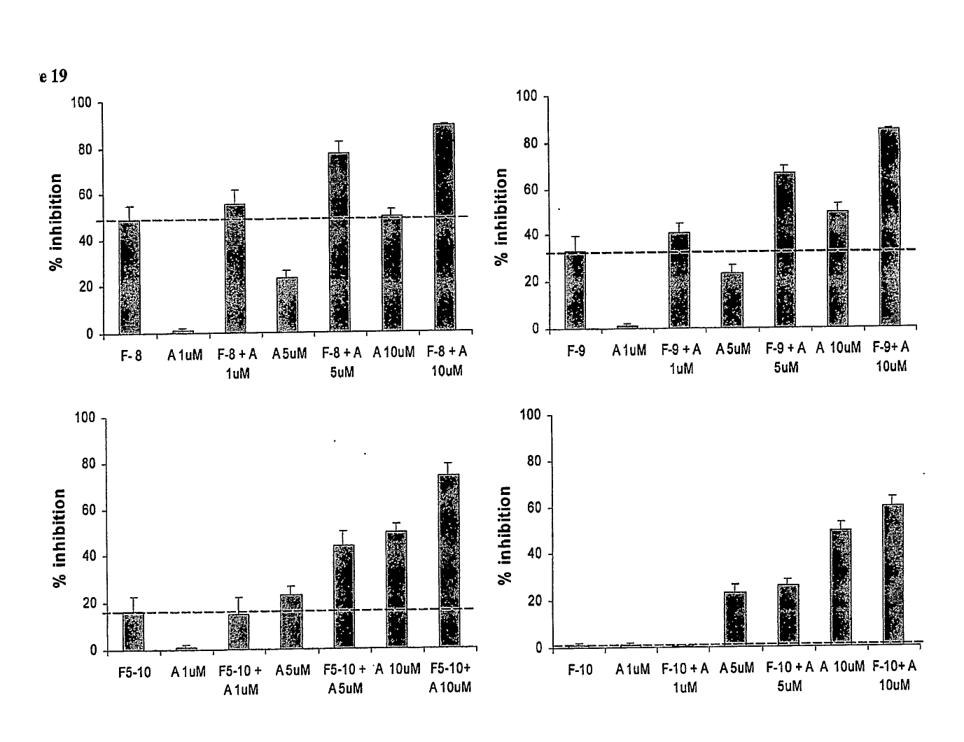

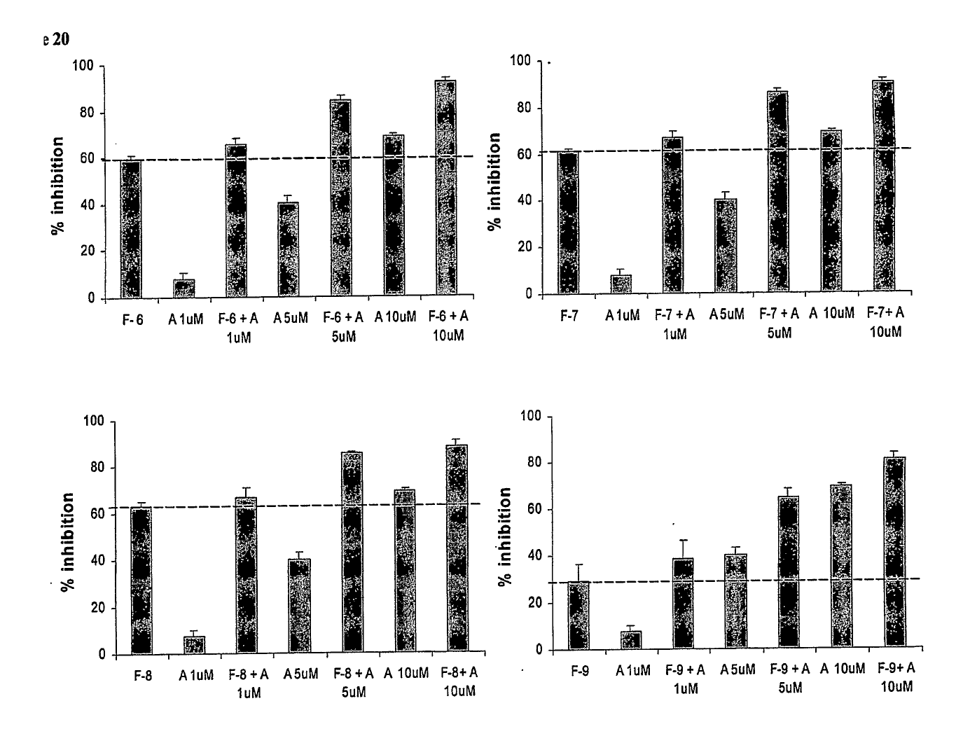

The combination of another NK-1 receptor antagonist (L-733,060) with dexamethasone also enhances the efficacy of dexamethasone by lowering the concentration of the anti-inflammatory agent required for a similar inhibitory effect (FIG. 18). Reciprocally, the combination of another glucocorticoid such as fluticasone (FIG. 19) or flunisolide (FIG. 20) with the NK-1 receptor antagonist L-703,606 also enhances the efficacy of dexamethasone by lowering the concentration of the anti-inflammatory agent required for a similar inhibitory effect.

### Example 4

### Combination of an NK-1 Receptor Antagonist and an Immunosuppressor

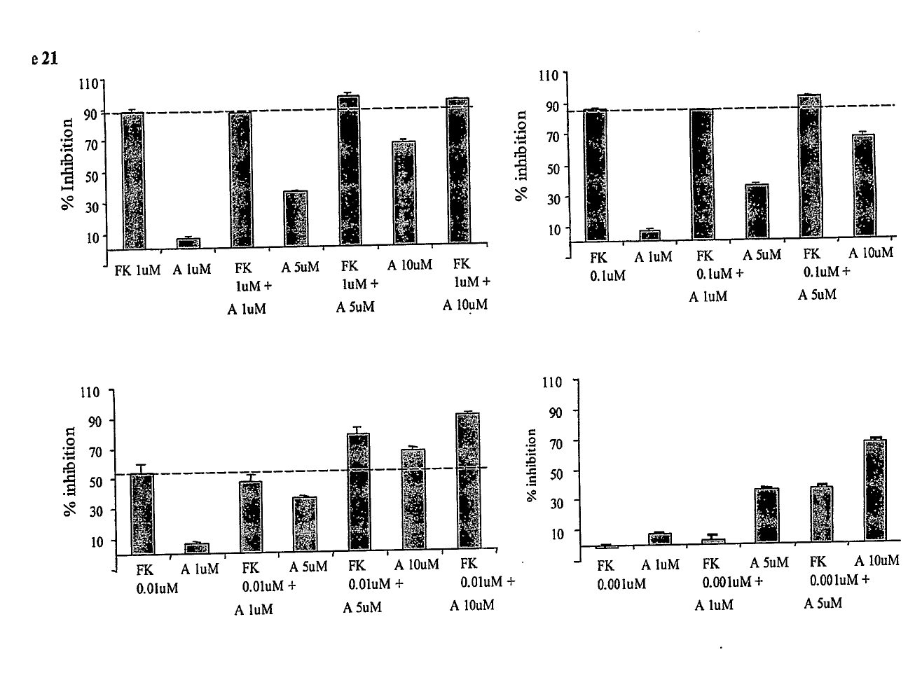

As noted above, the combination of neurokinin-1 receptor antagonists and an immunosuppressor agent is useful for the treatment of a wide variety of diseases, disorders and conditions associated with metachromatic cell activation (e.g. allergy, urticaria, etc.). Desirably, an immunosuppressor inhibits the calcium-calmodulin-activated serine/threonine-specific protein phosphatase calcineurin. Our data indicate that Tacrolimus (Sigma, # F4679) is a potent blocker of IgE-dependent activation and degranulation of metachromatic cells (FIG. 21). The structure of Tacrolimus is provided below.

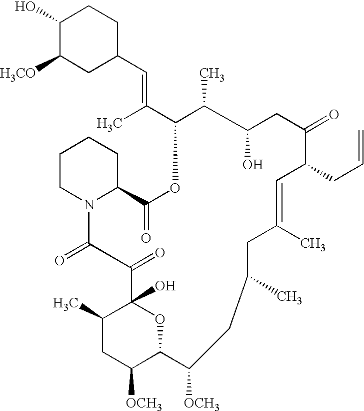

As an illustration of the efficacy of the combinations encompassed by the present invention, our data indicate that neurokinin-1 receptor antagonist L-703,606 (10 μM) potentiates the inhibitory effects of immunosuppressor agent Tacrolimus at the concentration of 0.01 μM on antigen-induced maximal cell degranulation (FIG. 21).

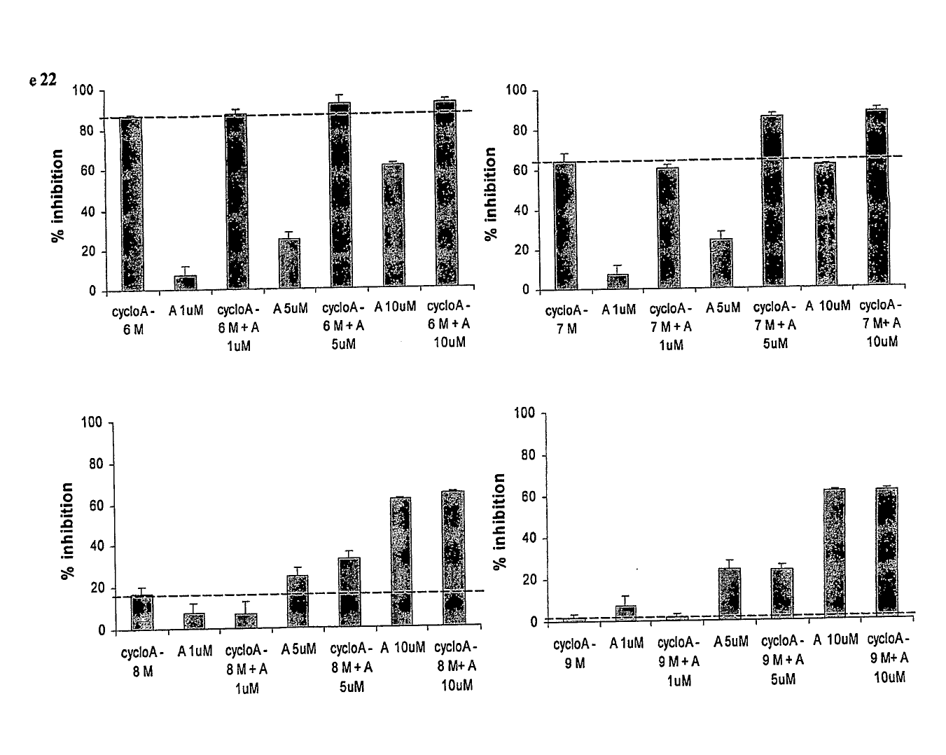

Our data also indicate that cyclosporin A (SigmaAldrich # 30024; structure provided below) is a potent blocker of IgE-dependent activation and degranulation of metachromatic cells (FIG. 22).

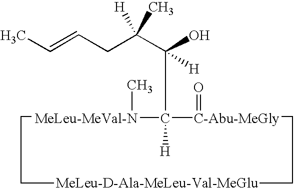

Our data indicate that neurokinin-1 receptor antagonist L-703,606 (10 μM) potentiates the inhibitory effect of the immunosuppressor cyclosporin A (SigmaAldrich # 30024) at the concentration of 0.1 μM on antigen-induced maximal cell degranulation (FIG. 22).

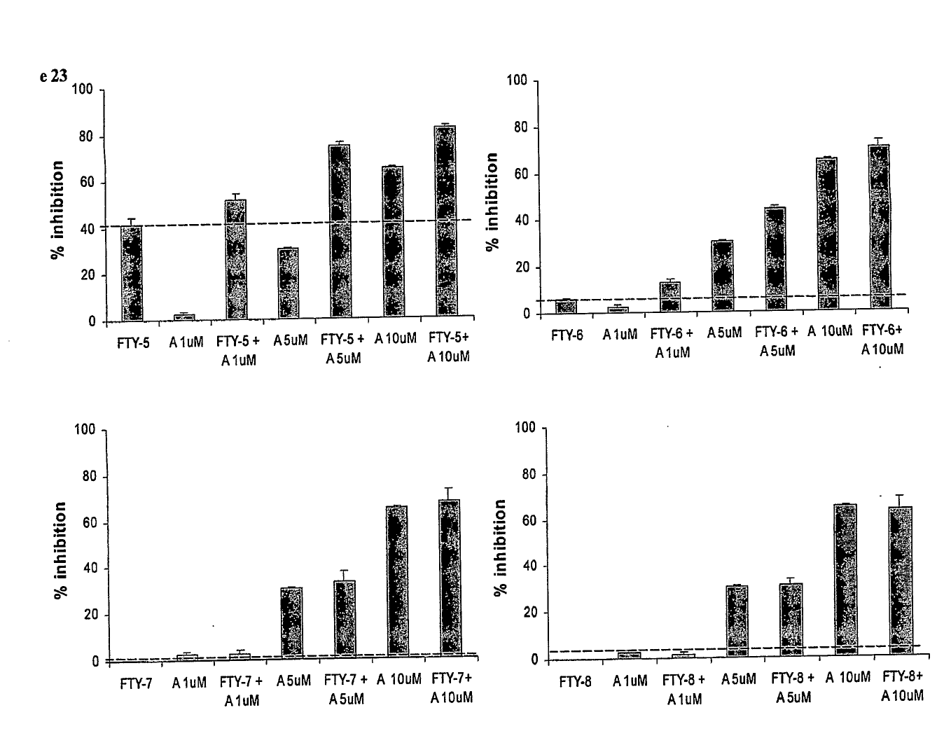

Our data further indicate that FTY720 (Cayman # 1006292; structure provided below) is a potent blocker of IgE-dependent activation and degranulation of metachromatic cells (FIG. 23).

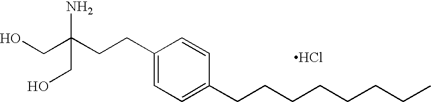

As another illustration of the efficacy of the drug combinations encompassed by the present invention, our data indicate that neurokinin-1 receptor antagonist L-703,606 (1, 5 and 10 μM) potentiates the inhibitory effect of immunosuppressor FTY720 at the concentration of 5 μM on antigen-induced maximal cell degranulation (FIG. 23).

### Example 5

### Combination of an NK-1 Receptor Antagonist and a Kinase Inhibitor

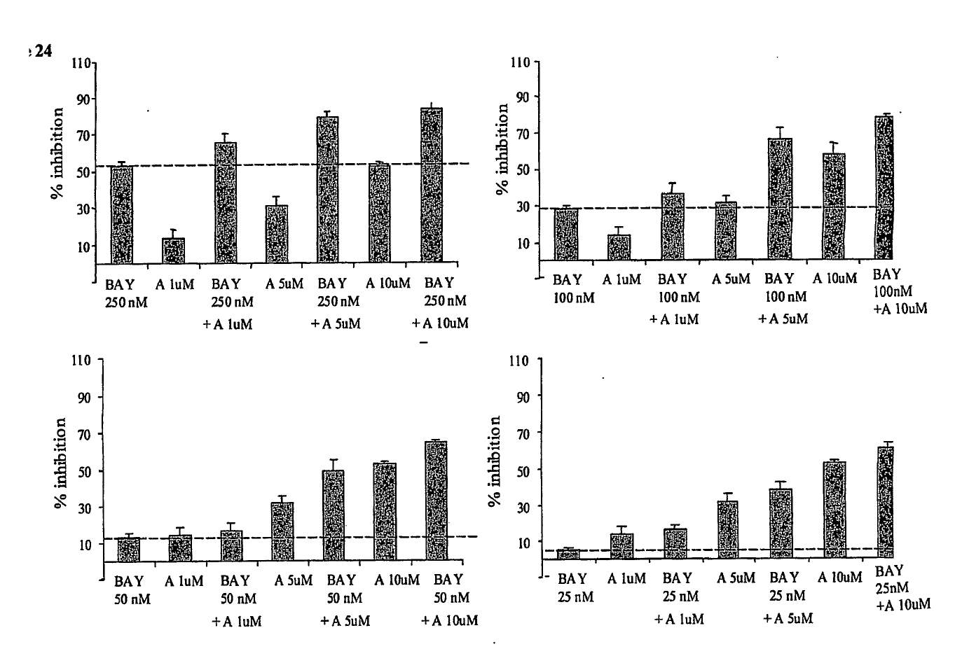

As noted above, the combination of a neurokinin-1 receptor antagonist and an inhibitor of a kinase involved in metachromatic cell activation and degranulation is useful for the treatment of a wide variety of diseases, disorders and conditions associated with metachromatic cell activation (e.g., allergy, urticaria, etc.). Our data confirm that the Syk kinase inhibitor BAY 61-3606 (SigmaAldrich # B9685; structure provided below) is a potent blocker of IgE-dependent activation and degranulation of metachromatic cells (FIG. 24).

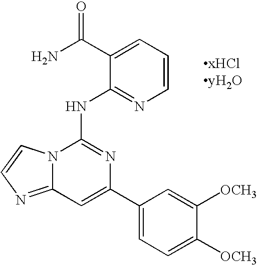

As an illustration of the effectiveness of a combination of a neurokinin-1 receptor antagonist with a kinase inhibitor in inhibiting metachromatic cell activation, we showed that L-703,606 at the concentration of 5 and 10 μM potentiates the inhibitory effects of Syk kinase inhibitor BAY 61-3606 at the concentration of 100 and 250 nM on antigen-induced maximal cell degranulation (FIG. 24).

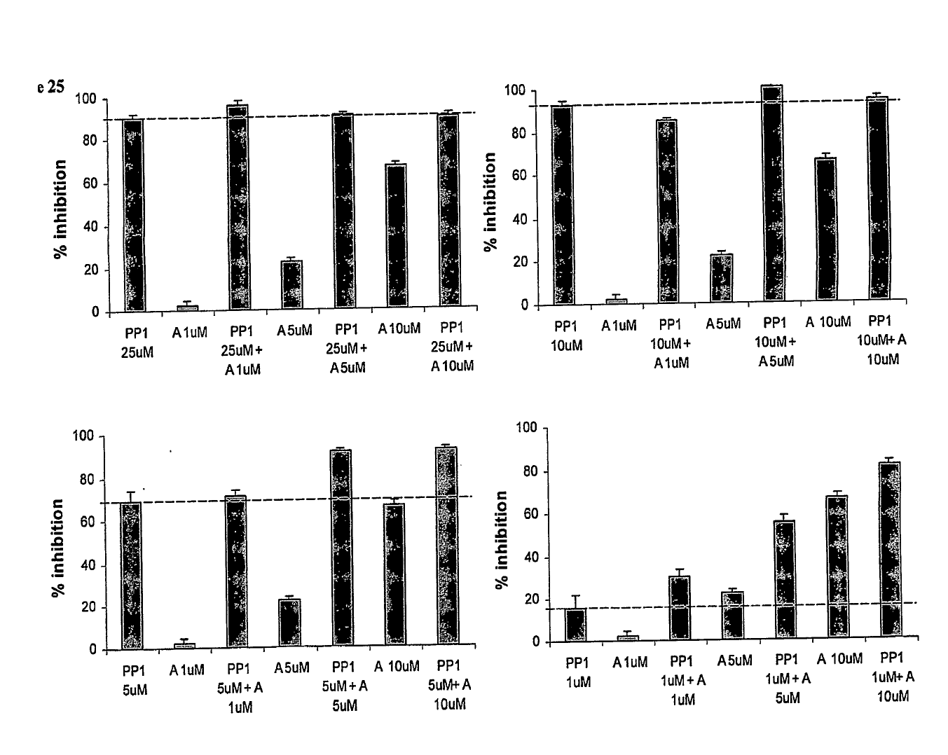

Our data confirm that the Src kinase family inhibitor PP1 (Biomol Int. # EI2750001; structure provided below) is a potent blocker of IgE-dependent activation and degranulation of metachromatic cells (FIG. 25).

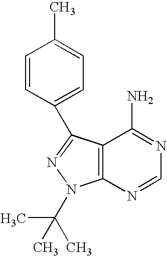

As an illustration of the effectiveness of a combination of a neurokinin-1 receptor antagonist with a kinase inhibitor in inhibiting metachromatic cell activation, we showed that L-703,606 at the concentration of 5 and 10 μM potentiates the inhibitory effect of Src kinase family inhibitor PP1 at the concentration of 1 and 5 μM on antigen-induced maximal cell degranulation (FIG. 25).

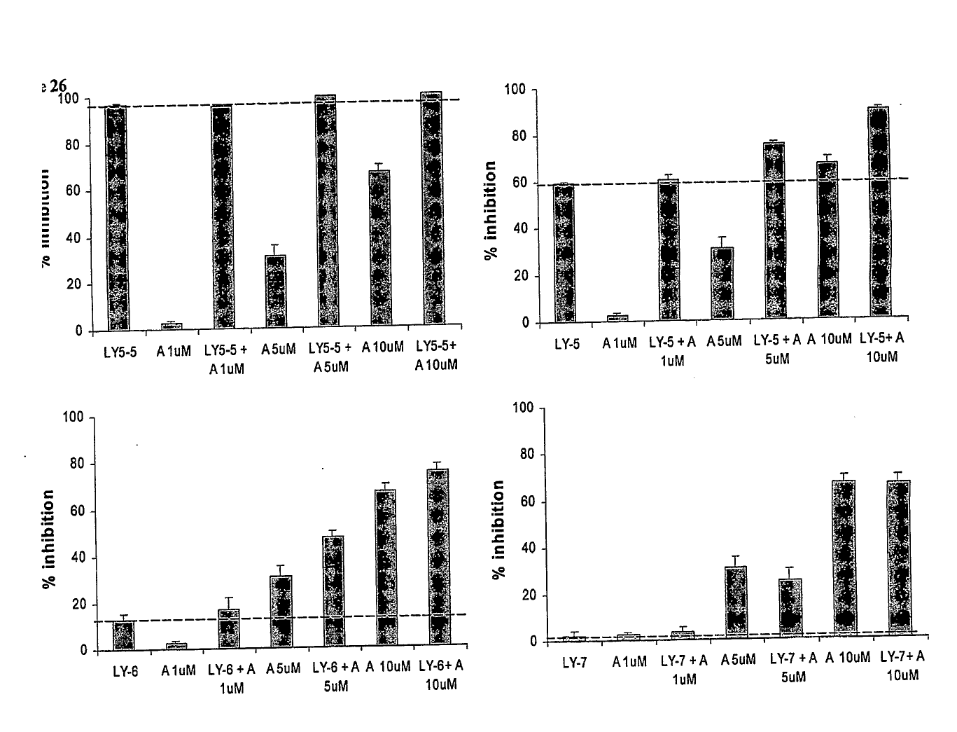

Our data also confirm that the PI3K inhibitor LY-294,002 (SigmaAldrich # L9908; structure provided below) is a potent blocker of IgE-dependent activation and degranulation of metachromatic cells (FIG. 26).

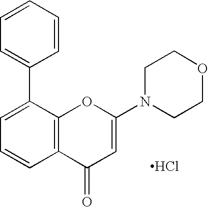

As an illustration of the effectiveness of a combination of a neurokinin-1 receptor antagonist with a kinase inhibitor in inhibiting metachromatic cell activation, we showed that L-703,606 at the concentration of 5 and 10 μM potentiates the inhibitory effect of PI3K inhibitor LY-294,002 at the concentration of 1 and 10 μM on antigen-induced maximal cell degranulation (FIG. 26).

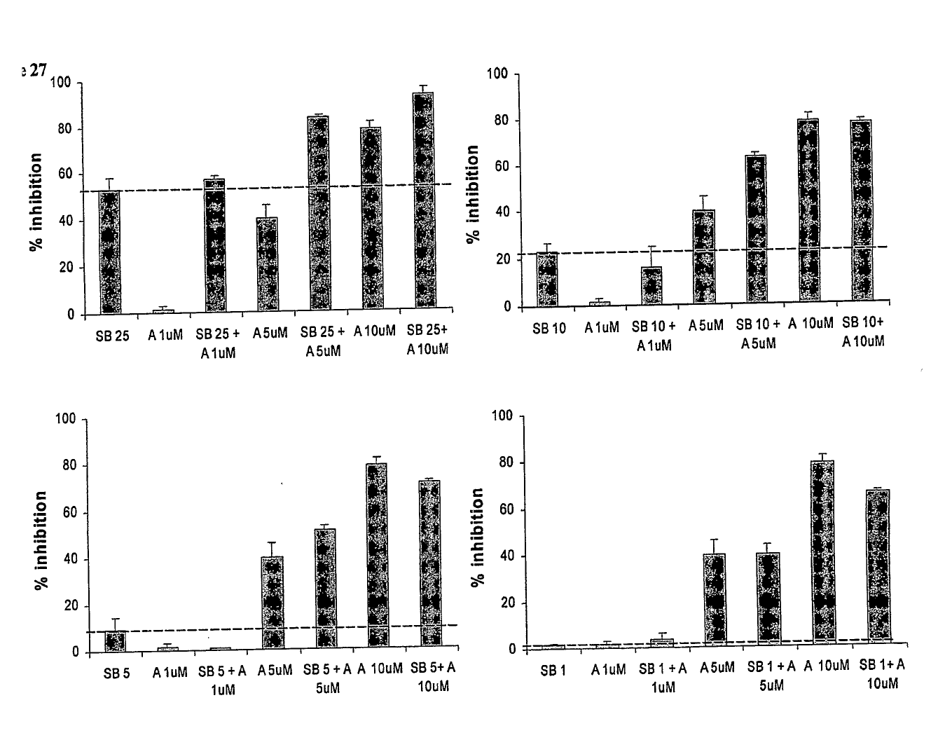

Our data further confirm that the p38 kinase inhibitor SB202190 (Sigma Aldrich # S70670; structure provided below) is a potent blocker of IgE-dependent activation and degranulation of metachromatic cells (FIG. 27).

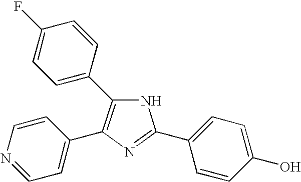

As an illustration of the effectiveness of a combination of a neurokinin-1 receptor antagonist with a kinase inhibitor in inhibiting metachromatic cell activation, we showed that L-703,606 at the concentration of 5 and 10 μM potentiates the inhibitory effect of p38 kinase inhibitor SB202190 at the concentration of 10 and 25 μM on antigen-induced maximal cell degranulation (FIG. 27).

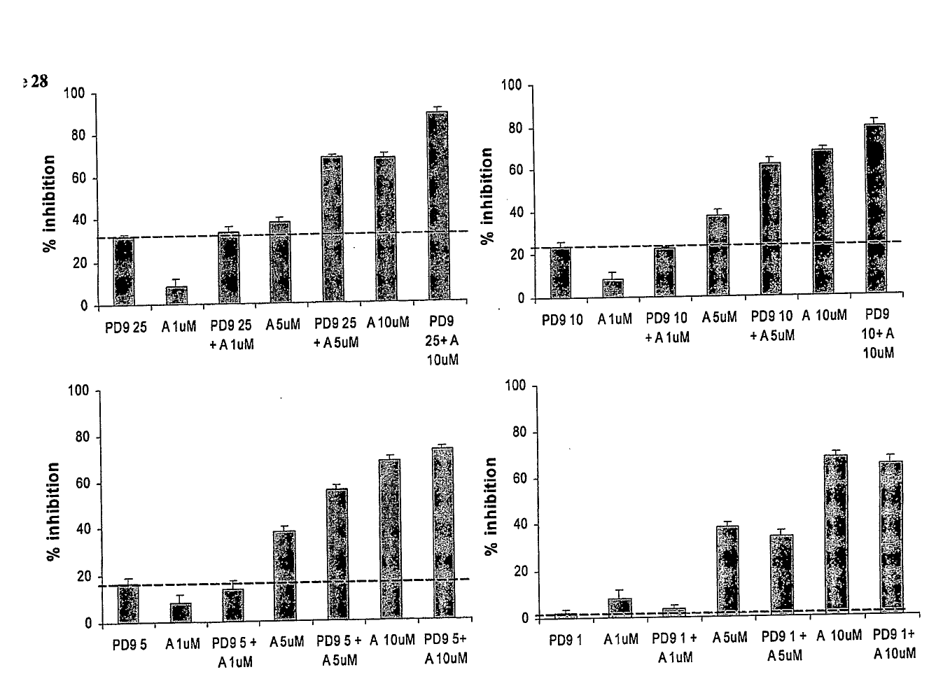

Our data also confirm that the MAPKK inhibitor PD98059 (CellSignaling # S70670; structure provided below) is a potent blocker of IgE-dependent activation and degranulation of metachromatic cells (FIG. 28).

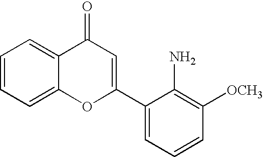

As an illustration of the effectiveness of a combination of a neurokinin-1 receptor antagonist with a kinase inhibitor in inhibiting metachromatic cell activation, we showed that L-703,606 at the concentration of 5 and 10 μM potentiates the inhibitory effect of MAPKK inhibitor PD98059 at the concentration of 5, 10 and 25 μM antigen-induced maximal cell degranulation (FIG. 28).

### Example 6

### Methods of Treatment Using an NK-1 Receptor Antagonist and an Inhibitor of Metachromatic Cell Activation

A therapeutically effective amount of a composition containing an NK-1 receptor antagonist and an inhibitor of metachromatic cell activation can be administered to a patient (e.g., a human) having or being at risk for acquiring a disorder associated with abnormal metachromatic cell activation or a disorder associated with a single nucleotide polymorphism isoform of the NK-1 receptor (e.g., GenBank Accession number BD223571). As such, a composition containing WIN 51,708, L-703,606 oxalate salt, L-733,060 hydrochloride, or Aprepitant and dexamethasone, fluticasone, flunisolide, or mometasone can be administered to a patient suffering from or being at risk of acquiring allergic or non-allergic rhinitis, asthma, or urticaria, transplant rejection, or an immuno-inflammatory disorder. Desirably, the combination contains Aprepitant and fluticasone; Aprepitant and budesonide; Aprepitant and Tacrolimus/FK506; Aprepitant and cyclosporin A; Aprepitant, budesonide, and formoterol; or Aprepitant, fluticasone, and salmeterol.

Other desirable combinations for treating a patient (e.g., a human) having or being at risk for acquiring a disorder associated with abnormal metachromatic cell activation or a disorder associated with a single nucleotide polymorphism isoform of the NK-1 receptor (e.g., GenBank Accession number BD223571) include the particular combinations of NK-1 receptor antagonists and immunsuppressors and/or kinase inhibitors described herein. As such, a combination for treatment of a disorder associated with abnormal metachromatic cell activation desirably contains L-703,606 and Cyclosporin A; L-703,606 and Tacrolimus/FK506; L-703,606 and FTY720; L-703,606 and BAY 61-3606; L-703,606 and PP1; L-703,606 and LY-294,002; or L-703,606 and PD98059. A further desirable combination is a beta-2 adrenergic receptor agonist such as indacaterol and a glucocorticoid such as mometasone alone or in combination with the compounds described herein.

All patents, patent applications, patent application publications, and other publications cited or referred to in this specification are herein incorporated by reference to the same extent as if each independent patent, patent application, patent application publication or publication was specifically and individually indicated to be incorporated by reference.

What is claimed is:

1.-123. (canceled)

124. A composition comprising an NK-1 receptor inhibitor and an anti-inflammatory compound, wherein the NK-1 receptor inhibitor is not a compound encompassed by Formula I, Aprepitant, or Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide).

125. The composition of claim 124, wherein said NK-1 receptor inhibitor is selected from the group consisting of RP 67580, WIN 51078, L-733,060, L-703,606, MDL 105,212, Antagonist D, R116301, CGP49823, CP-96345, CP-99994, GR-203040, MDL-103392, L-760735, SDZ-NKT-343, nolpitanitium (SR-140333), AV608, LY686017, E-6006, Vestipitant, 823296, Netupitant, H1/NK1 Dual Antagonists, MPC-4505, CP-122721, CJ-12,255, SRR240600, and TA-5538.

126. The composition of claim 124, wherein said anti-inflammatory compound is a steroid.

127. The composition of claim 126, wherein said steroid is dexamethasone, fluticasone, flunisolide, budesonide, or mometasone.

128. A composition comprising (i) an NK-1 receptor inhibitor, (ii) an inhibitor of metachromatic cell activation, and (iii) a beta-2 adrenergic receptor agonist.

129. The composition of claim 128, wherein said inhibitor of metachromatic cell activation is fluticasone and said beta-2 adrenergic receptor agonist is salmeterol, said inhibitor of metachromatic cell activation is budesonide and said beta-2 adrenergic receptor agonist is formoterol, or said inhibitor of metachromatic cell activation is mometasone and said beta-2 adrenergic receptor agonist is indacaterol.

130. The composition of claim 128, wherein said NK-1 receptor inhibitor is Aprepitant.

131. The composition of claim 124, wherein said composition is in a pharmaceutically acceptable carrier.

132. The composition of claim 128, wherein said composition is in a pharmaceutically acceptable carrier.

133. A pharmaceutically acceptable composition comprising the composition of claim 124.

134. A pharmaceutically acceptable composition comprising the composition of claim 128.

135. A kit comprising the composition of claim 124 and instructions for administration of said composition to a subject.

136. A kit comprising the composition of claim 128 and instructions for administration of said composition to a subject.

137. A method of treating a disease, disorder, or condition associated with metachromatic cell activation in a subject, said method comprising administering to a subject in need thereof a therapeutically effective amount of a composition comprising an NK-1 receptor inhibitor and an inhibitor of metachromatic cell activation, wherein the NK-1 receptor inhibitor is not a compound encompassed by Formula I, or Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide).

138. The method of claim 137, wherein said NK-1 receptor inhibitor is selected from the group consisting of RP 67580, WIN 51078, L-733,060, L-703,606, MDL 105,212, Antagonist D, R116301, CGP49823, CP-96345, CP-99994, GR-203040, MDL-103392, L-760735, SDZ-NKT-343, nolpitanitium (SR-140333), AV608, LY686017, E-6006, Vestipitant, 823296, Netupitant, H1/NK1 Dual Antagonists, MPC-4505, CP-122721, CJ-12,255, SRR240600, and TA-5538.

139. The method of claim 137, wherein said inhibitor of metachromatic cell activation is an anti-inflammatory compound.

140. The method of claim 139, wherein said anti-inflammatory compound is a steroid.

141. The method of claim 140, wherein said steroid is dexamethasone, fluticasone, flunisolide, budesonide, or mometasone.

142. A method of treating a disorder selected from the group consisting of allergic or non-allergic rhinitis, allergic or non-allergic asthma, allergic or non-allergic urticaria, an immuno-inflammatory disorder, an autoimmune disorder, and transplant rejection in a subject, said method comprising administering to a subject in need thereof a therapeutically effective amount of a composition comprising an NK-1 receptor inhibitor and an inhibitor of metachromatic cell activation, wherein the NK-1 receptor inhibitor is not a compound encompassed by Formula I.

143. The method of claim 142, wherein said disorder is allergic or non-allergic rhinitis or allergic or non-allergic asthma.

144. The method of claim 142, wherein said NK-1 receptor inhibitor is selected from the group consisting of RP 67580, WIN 51078, L-733,060, L-703,606, MDL 105,212, Antagonist D, Aprepitant, R116301, CGP49823, CP-96345, CP-99994, GR-203040, MDL-103392, L-760735, SDZ-NKT-343, nolpitanitium (SR-140333) AV608, LY686017, E-6006, Casopitant/GW679769 ((2R,4S)-4-(4-acetylpiperazin-1-yl)-N-{(1R)-1-[3,5-bis(trifluoromethyl)=phenyl]ethyl}-2-(4-fluoro-2-methylphenyl)-N-methylpiperidine-1-carboxamide), Vestipitant, 823296, Netupitant, H1/NK1 Dual Antagonists, MPC-4505, CP-122721, CJ-12,255, SRR240600, and TA-5538.

145. The method of claim 142, wherein said inhibitor of metachromatic cell activation is an anti-inflammatory compound.

146. The method of claim 145, wherein said anti-inflammatory compound is dexamethasone, fluticasone, flunisolide, budesonide, or mometasone.

147. The method of claim 142, wherein said subject is a mammal.

148. The method of claim 147, wherein said mammal is a human.

149. The method of claim 142, wherein said composition is in a pharmaceutically acceptable carrier.
